(19) United States
(12) Patent Application Publication
(10) Pub. No.: US 2010/0132073 A1
(43) Pub. Date: May 27, 2010

(54) SESQUITERPENOID MODIFYING ENZYMES

(75) Inventors: Lorenzo Caputi, York (GB); Eng-Kiat Lim, York (GB); Dianna Joy Bowles, York (GB)

Correspondence Address: SPECKMAN LAW GROUP PLLC, 1201 THIRD AVENUE, SUITE 330, SEATTLE, WA, 98101, US

(73) Assignee: THE UNIVERSITY OF YORK, York (GB)

(21) Appl. No.: 12/517,109

(22) PCT Filed: Nov. 26, 2007
(86) PCT No.: PCT/GB07/04518

§ 371 Date: Jan. 21, 2010

(30) Foreign Application Priority Data
Dec. 1, 2006 (GB) 0624067.5
Dec. 20, 2006 (GB) 0625302.5

(51) Int. Cl. A01H 5/00; C12N 1/00; C12P 19/18; C07H 15/20; C07H 15/02; A61K 31/7028; C07H 1/08; A61P 35/00; A61P 31/04; A61P 31/10

(52) U.S. Cl. 800/298; 435/243; 435/97; 800/295; 536/4.1; 514/25; 536/128

(57) ABSTRACT

We describe glycosyltransferase polypeptides that modify sesquiterpenoids and including pharmaceutical compositions comprising glycosylated sesquiterpenoids; methods to treat diseases, in particular cancer, bacterial and fungal infections and also flavourings and scents comprising glycosylated sesquiterpenoids.

The invention relates to glycosyltransferase polypeptides that modify sesquiterpenoids and including pharmaceutical compositions comprising glycosylated sesquiterpenoids; methods to treat diseases, in particular cancer, bacterial and fungal infections and also flavourings and scents comprising glycosylated sesquiterpenoids.

Plant terpenoids, also called isoprenoids are products derived from a five carbon isoprene unit and have diverse activities that include anti-cancer and anti-microbial activity. They are also used to flavour and/or scent a variety of commercial products. Terpenoids are classified with reference to the number of isoprene units that comprise the particular terpenoid. For example a monoterpenoid comprises two isoprene units; a sesquiterpenoid comprises three isoprene units and a di-terpenoid four isoprene units. Polyterpnoids comprise multiple isoprene units. There are many thousands of examples of terpenoids.

Artemisinin is a sesquiterpene lactone endoperoxide and is a natural product produced by the plant Artemesia annua. Artemisinin has long been known to have anti-malarial activity and is typically used in combination with anti-malarial therapeutics, for example lumefantrine, mefloquine, amodiaquine, suffadoxine, chloroquine, in artemisinin combination therapies (ACT). Artemisinin is only produced by the plant under certain conditions and is isolated from leaves. The in planta synthesis of artemisinin is via the mevalonate pathway and is a multi-step process that results in the formation of artemisinic acid which is converted to artemisinin. In addition to its anti-malarial activity artemisinin has also been shown to have anti-cancer activity with respect to melanoma, see EP1 658 844 and U.S. Pat. No. 5,219,880.

Artemisinic acid is a biologically active molecule although not as an anti-malarial compound per se. It has been shown to have anti-bacterial and anti-fungal activities see Dhingra et al Current Science (2000) 78(6): 709, and also anti-cancer activity. Artemisinic acid is far more abundant than artemisinin in Artemesia annua and therefore a great deal of effort has been dedicated to the purification of artemisinic acid from plants as a precursor that can then be chemically converted to artemisinin. For example, U.S. Pat. No. 4,992,561 describes a process for the conversion of artemisinic acid to artemisinin by modification of artemisinic acid to dihydroartemisinic acid by reduction of the exocyclic methylene group. Dihydroartemisinic acid is then oxidised in two successive steps to artemisinin.

An alternative to purification of artemisinic acid from a plant source is the genetic engineering of microbial cells to reproduce the metabolic pathway for the synthesis of artemisinin intermediates. The final steps in the production of artemisinin are the conversion of farnesyl diphosphate to amorpha-4,11-diene (amorphadeine) by amorphadeine synthase followed by conversion of amorphadeine to artemisinic acid by a cytochrome P450 monooxygenase. There then follows two photooxidation steps to the conversion of artemisinic acid to artemisinin.

An example of engineering microbial cells is provided in Martin et al Nature Biotechnology (2003) 21(7): 796-802, which is incorporated by reference in its entirety. This describes the engineering of the mevalonate pathway in E. coli for the production of terpenoids. The manuscript describes the transformation of a bacterial host cell with a synthetic amporpha-4,11-diene synthase gene and the mevalonate pathway from Saccharomyces cerevisae. The transformed bacterial cells produce significant amounts of amorphadiene. Ro et al Nature (2006) 440: 940-943, the content of which is incorporated by reference in its entirety, describes the engineering of Saccharomyces cerevisae with amorphadeine synthase and the nucleic acid sequence of a cytochrome P450 monooxygenase that perform the three step conversion of amorphadeine to artemisinic acid. WO2005/033287, the content of which is incorporated by reference in its entirety, discloses much of the content of Martin et al and Ro et al. In addition WO00/12725, the content of which is incorporated by reference in its entirety, discloses an isolated nucleic acid molecule that encodes amorphadeine synthase from Artemesia annua.

A further example of a sesquiterpenoid is farnesol which is a flavouring added to many foods. It has also been shown to have pesticide activity, particularly to mites.

This disclosure relates to the identification of plant glycosyltransferases that glycosylate sesquiterpenoids to provide a glycosylated sesquiterpenoid ester. We describe bacterial cells that are genetically modified to include nucleic acid molecules and the glycosylation of sesquiterpenoids in a whole cell bioreactor. We also disclose the anti microbial activity of glycosylated sesquiterpenoids.

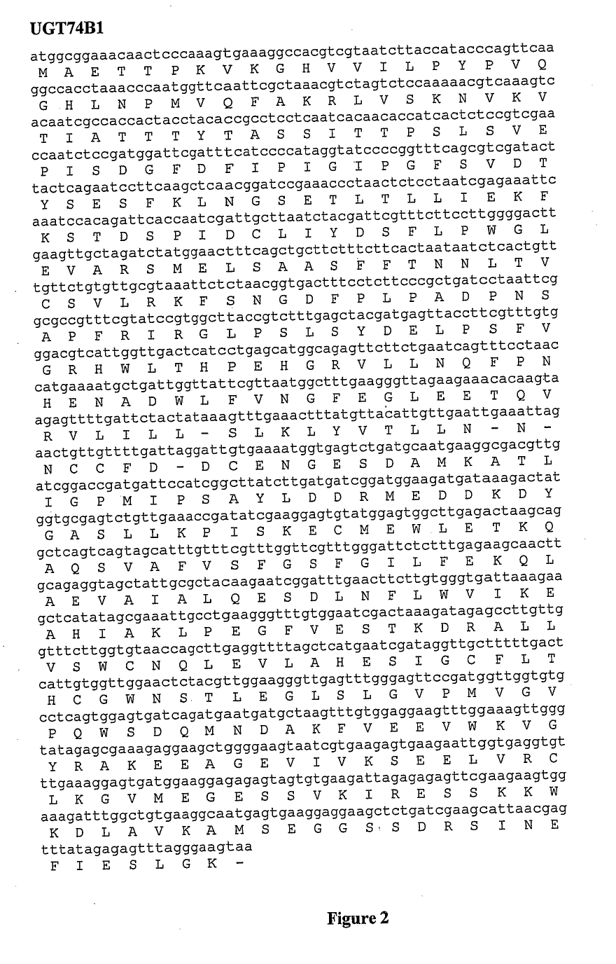

According to an aspect of the invention there is provided a microbial cell wherein the microbial cell is transformed with a vector that includes a nucleic acid molecule selected from the group consisting of:
i) a nucleic acid molecule comprising a nucleic acid sequence as represented in FIG. 2, 3, 4, 5, 6, 7, 8, 9, 10, 11, 12, 18, 19, 20, 21, 22, 23 or 24;
ii) a nucleic acid molecule that hybridizes under stringent hybridization conditions to a nucleic acid molecule as represented in FIG. 2, 3, 4, 5, 6, 7, 8, 9, 10, 11, 12, 18, 19, 20, 21, 22, 23 or 24; and that encodes a glycosyltransferase that glycosylates a sesquiterpenoid;
iii) a nucleic acid molecule that encodes a polypeptide comprising an amino acid sequence as represented in FIG. 2, 3, 4, 5, 6, 7, 8, 9, 10, 11, 12, 18, 19, 20, 21, 22, 23 or 24 ;

Hybridization of a nucleic acid molecule occurs when two complementary nucleic acid molecules undergo an amount of hydrogen bonding to each other. The stringency of hybridization can vary according to the environmental conditions surrounding the nucleic acids, the nature of the hybridization method, and the composition and length of the nucleic acid molecules used. Calculations regarding hybridization conditions required for attaining particular degrees of stringency are discussed in Sambrook et al., Molecular Cloning: A Laboratory Manual (Cold Spring Harbor Laboratory Press, Cold Spring Harbor, N.Y., 2001); and Tijssen, Laboratory Techniques in Biochemistry and Molecular Biology—Hybridization with Nucleic Acid Probes Part I, Chapter 2 (Elsevier, N.Y., 1993). The Tₘ is the temperature at which 50% of a given strand of a nucleic acid molecule is hybridized to its complementary strand. The following is an exemplary set of hybridization conditions and is not limiting:

Very High Stringency (Allows Sequences that Share at Least 90% Identity to Hybridize)
Hybridization: 5×SSC at 65° C. for 16 hours
Wash twice: 2×SSC at room temperature (RT) for 15 minutes each
Wash twice: 0.5×SSC at 65° C. for 20 minutes each

High Stringency (Allows Sequences that Share at Least 80% Identity to Hybridize)
Hybridization: 5×-6×SSC at 65° C.-70° C. for 16-20 hours
Wash twice: 2×SSC at RT for 5-20 minutes each
Wash twice: 1×SSC at 55° C.-70° C. for 30 minutes each

Low Stringency (Allows Sequences that Share at Least 50% Identity to Hybridize)
Hybridization: 6×SSC at RT to 55° C. for 16-20 hours
Wash at least twice: 2×-3×SSC at RT to 55° C. for 20-30 minutes each.

In preferred embodiment of the invention said nucleic acid molecule consists of a nucleic acid sequence as represented in FIG. 2, 3, 4, 5, 6, 7, 8, 9, 10, 11, or 12.

In a further preferred embodiment of the invention said microbial cell is transformed with a nucleic acid molecule that encodes a polypeptide wherein said polypeptide is an amorphadeine synthase that catalyses the conversion of farnesyl diphosphate to amorpha-4,11-diene.

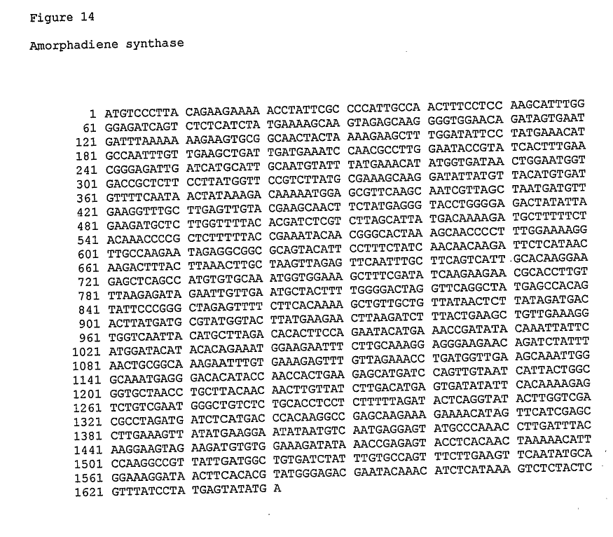

In a preferred embodiment of the invention said nucleic acid molecule comprises a nucleic acid sequence as represented in FIG. 14, or a nucleic acid molecule that hybridises to the nucleic acid molecule in FIG. 14 and encodes a polypeptide that is an amorphadeine synthase.

In a further preferred embodiment of the invention said microbial cell is transformed with a nucleic acid molecule that encodes a polypeptide wherein said polypeptide is a cytochrome P450 that catalyses the conversion of amorpha-4,11-diene to artemisinic acid.

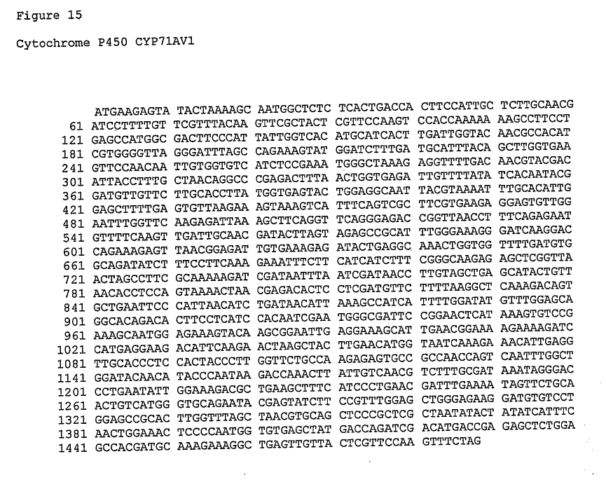

In a yet further preferred embodiment of the invention said nucleic acid molecule comprises a nucleic acid sequence as represented in FIG. 15, or a nucleic acid molecule that hybridises to the nucleic acid molecule in FIG. 15 and encodes a polypeptide that is a P450.

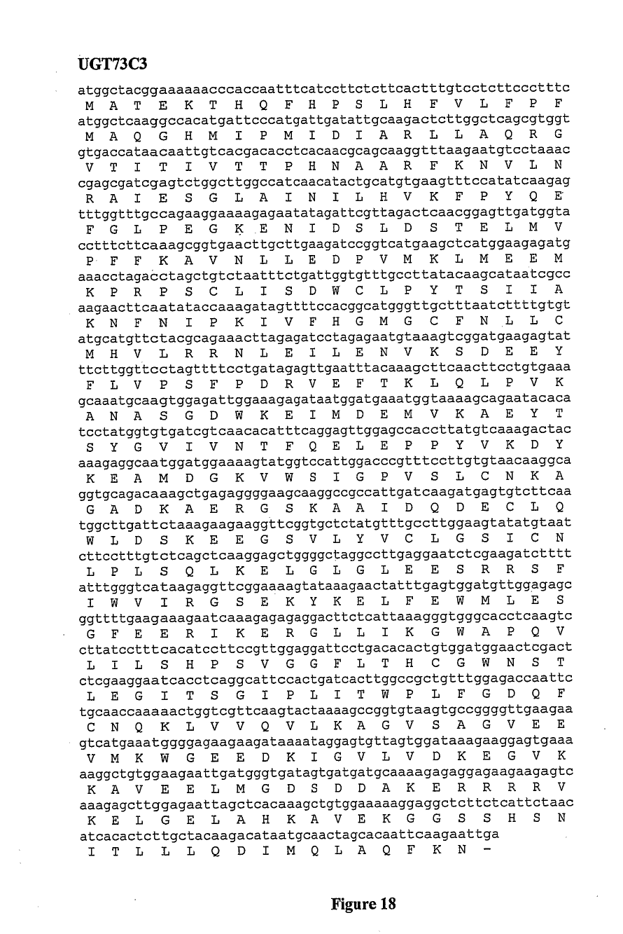

In a further preferred embodiment of the invention said nucleic acid molecule consists of a nucleic acid sequence as represented in FIG. 18, 19, 20, 21, 22, 23 or 24.

In a preferred embodiment of the invention said microbial cell is a bacterial cell.

In an alternative preferred embodiment of the invention said microbial cell is a yeast cell.

In a preferred embodiment of the invention said vector is an expression vector and said nucleic acid molecule encoding said glycosyltransferase is operably linked to a promoter.

A vector including nucleic acid (s) according to the invention need not include a promoter or other regulatory sequence, particularly if the vector is to be used to introduce the nucleic acid into cells for recombination into the genome for stable transfection.

Preferably the nucleic acid in the vector is operably linked to an appropriate promoter or other regulatory elements for transcription in a host cell such as a prokaryotic, (e.g. bacterial), or eukaryotic (e.g. fungal, plant, mammalian or insect cell). The vector may be a bi-functional expression vector which functions in multiple hosts. In the example of nucleic acids encoding polypeptides according to the invention this may contain its native promoter or other regulatory elements and in the case of cDNA this may be under the control of an appropriate promoter or other regulatory elements for expression in the host cell.

By “promoter” is meant a nucleotide sequence upstream from the transcriptional initiation site and which contains all the regulatory regions required for transcription. Suitable promoters include constitutive, tissue-specific, inducible, developmental or other promoters for expression in cells. Such promoters include viral, fungal, bacterial, animal and plant-derived promoters.

“Operably linked” means joined as part of the same nucleic acid molecule, suitably positioned and oriented for transcription to be initiated from the promoter. DNA operably linked to a promoter is “under transcriptional initiation regulation” of the promoter.

In a preferred embodiment the promoter is an inducible promoter or a developmentally regulated promoter.

Alternatively, or in addition, said vectors are vectors suitable for mammalian cell transfection or yeast cell transfection. In the latter example multi-copy vectors such as 2μ episomal vectors are preferred. Alternatively yeast CEN vectors and intergrating vectors such as YIP vectors are suitable for transformation of yeast species such as Saccharomyces cerevisiae and Pichia spp.

According to a further aspect of the invention there is provided the use of a cell according to the invention in the modification of a sequiterpenoid.

In a preferred embodiment of the invention said sequiterpenoid is artemisinic acid or farnesol.

According to a further aspect of the invention there is provided a process for the glycosylation of a sequiterpenoid comprising the steps of:
i) forming a preparation that includes a cell according to the invention and a sequiterpenoid;
ii) cultivating said preparation under conditions that allow the glycosylation of said sequiterpenoid with a sugar; and optionally
iii) isolating and purifying said glycosylated sequiterpenoid from said cell and/or the surrounding cell growth medium.

In a preferred method of the invention said sequiterpenoid is artemisinic acid or farnesol.

In a preferred method of the invention said cell is a bacterial cell.

In an alternative preferred method of the invention said cell is a yeast cell.

If microorganisms are used as organisms in the process according to the invention, they are grown or cultured in the manner with which the skilled worker is familiar, depending on the host organism. As a rule, microorganisms are grown in a liquid medium comprising a carbon source, usually in the form of sugars, a nitrogen source, usually in the form of organic nitrogen sources such as yeast extract or salts such as ammonium sulfate, trace elements such as salts of iron, manganese and magnesium and, if appropriate, vitamins, at temperatures of between 0° C. and 100° C., preferably between 10° C. and 60° C., while gassing in oxygen.

The pH of the liquid medium can either be kept constant, that is to say regulated during the culturing period, or not. The cultures can be grown batchwise, semi-batchwise or continuously. Nutrients can be provided at the beginning of the fermentation or fed in semi-continuously or continuously. The products produced can be isolated from the organisms as described above by processes known to the skilled worker, for example by extraction, distillation, crystallization, if appropriate precipitation with salt, and/or chromatography. To this end, the organisms can advantageously be disrupted beforehand. In this process, the pH value is advantageously kept between pH 4 and 12, preferably between pH 6 and 9, especially preferably between pH 7 and 8.

An overview of known cultivation methods can be found in the textbook by Chmiel (Bioprozeβtechnik 1. Einführung in die Bioverfahrenstechnik [Bioprocess technology 1. Introduction to Bioprocess technology] (Gustav Fischer Verlag, Stuttgart, 1991)) or in the textbook by Storhas (Bioreaktoren and periphere Einrichtungen [Bioreactors and peripheral equipment] (Vieweg Verlag, Brunswick/Wiesbaden, 1994)).

The culture medium to be used must suitably meet the requirements of the strains in question. Descriptions of culture media for various microorganisms can be found in the textbook “Manual of Methods for General Bacteriology” of the American Society for Bacteriology (Washington D.C., USA, 1981).

As described above, these media which can be employed in accordance with the invention usually comprise one or more carbon sources, nitrogen sources, inorganic salts, vitamins and/or trace elements.

Preferred carbon sources are sugars, such as mono-, di- or polysaccharides. Examples of carbon sources are glucose, fructose, mannose, galactose, ribose, sorbose, ribulose, lactose, maltose, sucrose, raffinose, starch or cellulose. Sugars can also be added to the media via complex compounds such as molasses or other by-products from sugar refining. The addition of mixtures of a variety of carbon sources may also be advantageous. Other possible carbon sources are oils and fats such as, for example, soya oil, sunflower oil, peanut oil and/or coconut fat, fatty acids such as, for example, palmitic acid, stearic acid and/or linoleic acid, alcohols and/or polyalcohols such as, for example, glycerol, methanol and/or ethanol, and/or organic acids such as, for example, acetic acid and/or lactic acid.

Nitrogen sources are usually organic or inorganic nitrogen compounds or materials comprising these compounds. Examples of nitrogen sources comprise ammonia in liquid or gaseous form or ammonium salts such as ammonium sulfate, ammonium chloride, ammonium phosphate, ammonium carbonate or ammonium nitrate, nitrates, urea, amino acids or complex nitrogen sources such as cornsteep liquor, soya meal, soya protein, yeast extract, meat extract and others. The nitrogen sources can be used individually or as a mixture.

Inorganic salt compounds which may be present in the media comprise the chloride, phosphorus and sulfate salts of calcium, magnesium, sodium, cobalt, molybdenum, potassium, manganese, zinc, copper and iron.

Inorganic sulfur-containing compounds such as, for example, sulfates, sulfites, dithionites, tetrathionates, thiosulfates, sulfides, or else organic sulfur compounds such as mercaptans and thiols may be used as sources of sulfur for the production of sulfur-containing fine chemicals, in particular of methionine.

Phosphoric acid, potassium dihydrogenphosphate or dipotassium hydrogenphosphate or the corresponding sodium-containing salts may be used as sources of phosphorus.

Chelating agents may be added to the medium in order to keep the metal ions in solution. Particularly suitable chelating agents comprise dihydroxyphenols such as catechol or protocatechuate and organic acids such as citric acid.

The fermentation media used according to the invention for culturing microorganisms usually also comprise other growth factors such as vitamins or growth promoters, which include, for example, biotin, riboflavin, thiamine, folic acid, nicotinic acid, panthothenate and pyridoxine. Growth factors and salts are frequently derived from complex media components such as yeast extract, molasses, cornsteep liquor and the like. It is moreover possible to add suitable precursors to the culture medium. The exact composition of the media compounds heavily depends on the particular experiment and is decided upon individually for each specific case. Information on the optimization of media can be found in the textbook “Applied Microbiol. Physiology, A Practical Approach” (Editors P. M. Rhodes, P. F. Stanbury, IRL Press (1997) pp. 53-73, ISBN 0 19 963577 3). Growth media can also be obtained from commercial suppliers, for example Standard 1 (Merck) or BHI (brain heart infusion, DIFCO) and the like.

All media components are sterilized, either by heat (20 min at 1.5 bar and 121° C.) or by filter sterilization. The components may be sterilized either together or, if required, separately. All media components may be present at the start of the cultivation or added continuously or batchwise, as desired.

The culture temperature is normally between 15° C. and 45° C., preferably at from 25° C. to 40° C., and may be kept constant or may be altered during the experiment. The pH of the medium should be in the range from 5 to 8.5, preferably around 7.0. The pH for cultivation can be controlled during cultivation by adding basic compounds such as sodium hydroxide, potassium hydroxide, ammonia and aqueous ammonia or acidic compounds such as phosphoric acid or sulfuric acid. Foaming can be controlled by employing antifoams such as, for example, fatty acid polyglycol esters. To maintain the stability of plasmids it is possible to add to the medium suitable substances having a selective effect, for example antibiotics. Aerobic conditions are maintained by introducing oxygen or oxygen-containing gas mixtures such as, for example, ambient air into the culture. The temperature of the culture is normally 20° C. to 45° C. and preferably 25° C. to 40° C. The culture is continued until formation of the desired product is at a maximum. This aim is normally achieved within 10 to 160 hours.

The fermentation broths obtained in this way, in particular those comprising polyunsaturated fatty acids; usually contain a dry mass of from 7.5 to 25% by weight.

The fermentation broth can then be processed further. The biomass may, according to requirement, be removed completely or partially from the fermentation broth by separation methods such as, for example, centrifugation, filtration, decanting or a combination of these methods or be left completely in said broth. It is advantageous to process the biomass after its separation.

However, the fermentation broth can also be thickened or concentrated without separating the cells, using known methods such as, for example, with the aid of a rotary evaporator, thin-film evaporator, falling-film evaporator, by reverse osmosis or by nanofiltration.

According to a further aspect of the invention there is provided a transgenic plant wherein said plant is genetically modified by transfection with a vector that includes a nucleic acid molecule selected from the group consisting of:
i) a nucleic acid molecule comprising a nucleic acid sequence as represented in FIG. 2, 3, 4, 5, 6; 7, 8, 9, 10, 11, 12, 18, 19, 20, 21, 22, 23 or 24;
ii) a nucleic acid molecule that hybridizes under stringent hybridization conditions to a nucleic acid molecule as represented in FIG. 2, 3, 4, 5, 6; 7, 8, 9, 10, 11, 12, 18, 19, 20, 21, 22, 23 or 24; and that encodes a glycosyltransferase that glycosylates artemisinic acid;
iii) a nucleic acid molecule that encodes a polypeptide comprising an amino acid sequence as represented in FIG. 2, 3, 4, 5, 6; 7, 8, 9, 10, 11, 12, 18, 19, 20, 21, 22, 23 or 24.

In a preferred embodiment of the invention said plant is selected from the group consisting of: In a preferred embodiment of the invention said plant is selected from: corn (Zea mays), canola (Brassica napus, Brassica rapa ssp.), alfalfa (Medicago sativa), rice (Oryza sativa), rye (Secale cerale), sorghum (Sorghum bicolor, Sorghum vulgare), sunflower (helianthus annuas), wheat (Tritium aestivum), soybean (Glycine max), tobacco (Nicotiana tabacum), potato (Solanum tuberosum), peanuts (Arachis hypogaea), cotton (Gossypium hirsutum), sweet potato (Iopmoea batatus), cassava (Manihot esculenta), coffee (Cofea spp.), coconut (Cocos nucifera), pineapple (Anana comosus), citris tree (Citrus spp.) cocoa (Theobroma cacao), tea (Camellia senensis), banana (Musa spp.), avacado (Persea americana), fig (Ficus casica), guava (Psidium guajava), mango (Mangifer indica), olive (Olea europaea), papaya (Carica papaya), cashew (Anacardium occidentale), macadamia (Macadamia intergrifolia), almond (Prunus amygdalus), sugar beets (Beta vulgaris), oats, barley, vegetables and ornamentals.

Preferably, plants of the present invention are crop plants for example, cereals and pulses, maize, wheat, potatoes, tapioca, rice, sorghum, millet, cassava, barley, pea, and other root, tuber or seed crops and including peppermint and spearmint.

Important seed crops are oil-seed rape, sugar beet, maize, sunflower, soybean, and sorghum. Horticultural plants to which the present invention may be applied may include lettuce, endive, and vegetable brassicas including cabbage, broccoli, celery and cauliflower, and carnations and geraniums. The present invention may be applied in tobacco, cucurbits, carrot, strawberry, cherry, sunflower, tomato, pepper, and chrysanthemum.

Grain plants that provide seeds of interest include oil-seed plants and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mung bean, lima bean, fava been, lentils, chick pea.

In a preferred embodiment of the invention said plant is of the genus Artemesia spp; preferably Artemesia annua.

According to a further aspect of the invention there is provided a process for the glycosylation of a sesquiterpenoid comprising the steps of:
i) providing a transgenic plant or a seed transfected with a nucleic acid molecule comprising a nucleic acid sequence selected from the group consisting of:
   a) a nucleic acid sequence as represented in FIG. 2, 3, 4, 5, 6; 7, 8, 9, 10, 11, 12, 18, 19, 20, 21, 22, 23 or 24;
   b) a nucleic acid molecule that hybridizes under stringent hybridization conditions to a nucleic acid molecule as represented in 2, 3, 4, 5, 6; 7, 8, 9, 10, 11, 12, 18, 19, 20, 21, 22, 23 or 24 and that encodes a glycosyltransferase that glycosylates a sesquiterpenoid;
   c) a nucleic acid molecule that encodes a polypeptide comprising an amino acid sequence as represented in FIG. 2, 3, 4, 5, 6; 7, 8, 9, 10, 11, 12, 18, 19, 20, 21, 22, 23 or 24;
ii) cultivating said plant or seed under conditions that allow the glycosylation of a sesquiterpenoid with a sugar; and optionally
iii) isolating and purifying said glycosylated sesquiterpenoid from said plant and/or said seed.

According to an aspect of the invention there is provided an isolated sesquiterpenoid ester comprising a sugar pendant group to provide glycosylated sesquiterpenoid ester.

According to an aspect of the invention there is provided an isolated artemisinic acid ester comprising a sugar pendant group to provide glycosylated artemisinic acid.

In a preferred embodiment of the invention artemisinic acid has the structure

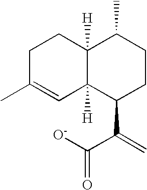

In a further preferred embodiment of the invention said glycosylated artemisinic acid is glycosylated with a glucose molecule.

In an alternative preferred embodiment of the invention said glycosylated artemisinic acid is glycosylated with a raffinose molecule.

In a further alternative embodiment of the invention said glycosylated artemisinic acid is glycosylated with a glucuronic acid molecule.

According to a further aspect of the invention there is provided glycosylated artemisinic acid for use as a pharmaceutical.

According to a further aspect of the invention there is provided a composition comprising a glycosylated artemisinic acid according to the invention. Preferably said composition is a pharmaceutical composition.

When administered, the compositions of the present invention are administered in pharmaceutically acceptable preparations. Such preparations may routinely contain pharmaceutically acceptable concentrations of salt, buffering agents, preservatives and compatible carriers.

The therapeutics of the invention can be administered by any conventional route, including injection or by gradual infusion over time. The administration may be, for example, oral, intravenous, intraperitoneal, intramuscular, intracavity, subcutaneous, or transdermal.

The compositions of the invention are administered in effective amounts. An “effective amount” is that amount of a composition that alone, or together with further doses, produces the desired response. In the case of treating a particular disease, such as cancer, the desired response is inhibiting the progression of the disease. This may involve only slowing the progression of the disease temporarily, although more preferably, it involves halting the progression of the disease permanently. This can be monitored by routine methods.

Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

The pharmaceutical compositions used in the foregoing methods preferably are sterile and contain an effective amount of glycosylated artemisinic acid for producing the desired response in a unit of weight or volume suitable for administration to a patient. The response can, for example, be measured by measuring the physiological effects of the composition, such as regression of a tumour, decrease of disease symptoms, modulation of apoptosis, etc.

The doses of glycosylated artemisinic acid administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

Other protocols for the administration of glycosylated artemisinic acid will be known to one of ordinary skill in the art, in which the dose amount, schedule of injections, sites of injections, mode of administration (e.g., intra-tumoural) and the like vary from the foregoing. Administration of glycosylated artemisinic acid compositions to mammals other than humans, (e.g. for testing purposes or veterinary therapeutic purposes), is carried out under substantially the same conditions as described above. A subject, as used herein, is a mammal, preferably a human, and including a non-human primate, cow, horse, pig, sheep, goat, dog, cat or rodent.

When administered, the pharmaceutical preparations of the invention are applied in pharmaceutically-acceptable amounts and in pharmaceutically-acceptable compositions. The term “pharmaceutically acceptable” means a non-toxic material that does not interfere with the effectiveness of the biological activity of the active ingredients. Such preparations may routinely contain salts, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

Glycosylated artemisinic acid compositions may be combined, if desired, with a pharmaceutically-acceptable carrier. The term “pharmaceutically-acceptable carrier” as used herein means one or more compatible solid or liquid fillers, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy.

The pharmaceutical compositions may contain suitable buffering agents, including: acetic acid in a salt; citric acid in a salt; boric acid in a salt; and phosphoric acid in a salt.

The pharmaceutical compositions also may contain, optionally, suitable preservatives, such as: benzalkonium chloride; chlorobutanol; parabens and thimerosal.

The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as syrup, elixir or an emulsion.

Compositions suitable for parenteral administration conveniently comprise a sterile aqueous or non-aqueous preparation of glycosylated artemisinic acid, which is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally-acceptable diluent or solvent. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono-or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa.

In a preferred embodiment of the invention said pharmaceutical composition is a cream adapted for topical application.

According to a further aspect of the invention there is provided a method to treat a fungal infection comprising administering an effective amount of glycosylated artemisinic acid according to the invention to an animal; preferably a human.

According to a further aspect of the invention there is provided a method to treat a bacterial infection comprising administering an effective amount of glycosylated artemisinic acid according to the invention to an animal; preferably a human.

In a preferred method of the invention said treatment is the topical application of; preferably glycosylated artemisinic acid is included in a cream.

According to a further aspect of the invention there is provided a method to treat a cancer comprising administering an effective amount of glycosylated artemisinic acid according to the invention to an animal; preferably a human.

As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “cancer” includes malignancies of the various organ systems, such as those affecting, for example, lung, breast, thyroid, lymphoid, gastrointestinal, and genito-urinary tract, as well as adenocarcinomas which include malignancies such as most colon cancers, renal-cell carcinoma, prostate cancer and/or testicular tumours, non-small cell carcinoma of the lung, cancer of the small intestine and cancer of the esophagus. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term “carcinoma” also includes carcinosarcomas, e.g., which include malignant tumours composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumours of mesenchymal derivation.

According to an aspect of the invention there is provided an isolated farnesol ester comprising a sugar pendant group to provide glycosylated farnesol.

In a preferred embodiment of the invention farnesol has the structure

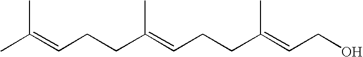

In a further preferred embodiment of the invention said glycosylated farnesol is glycosylated with a glucose molecule.

In an alternative preferred embodiment of the invention said glycosylated farnesol is glycosylated with a raffinose molecule.

In a further alternative embodiment of the invention said glycosylated farnesol is glycosylated with a glucuronic acid molecule.

According to a further aspect of the invention there is provided a composition comprising a glycosylated farnesol according to the invention.

According to a further aspect of the invention there is provided the use of glycosylated farnesol according to the invention as a flavouring.

According to a further aspect of the invention there is provided the use of glycosylated farnesol according to the invention as a food additive.

According to a further aspect of the invention there is provided the use of glycosylated farnesol according to the invention as a pesticide.

Throughout the description and claims of this specification, the words “comprise” and “contain” and variations of the words, for example “comprising” and “comprises”, means “including but not limited to”, and is not intended to (and does, not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

An embodiment of the invention will now be described by example only and with reference to the following figures:

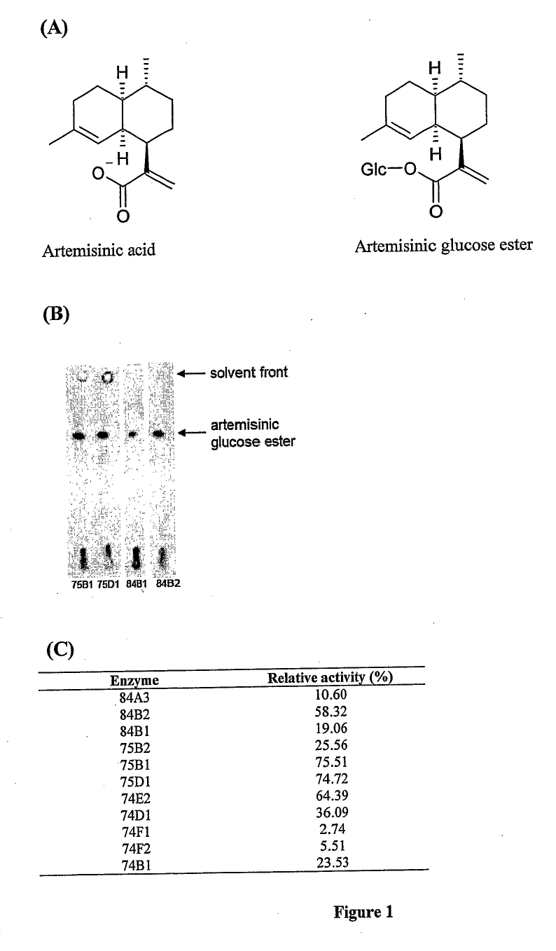

FIG. 1: (A) Chemical structures of artemisinic acid and artemisinic glucose ester. (B) TLC analysis of representative GTs capable of glucosylating artemisinic acid. (C) Relative activity of GTs towards artemisinic acid;

FIG. 2 Nucleic acid and amino acid sequence of UGT74B1;

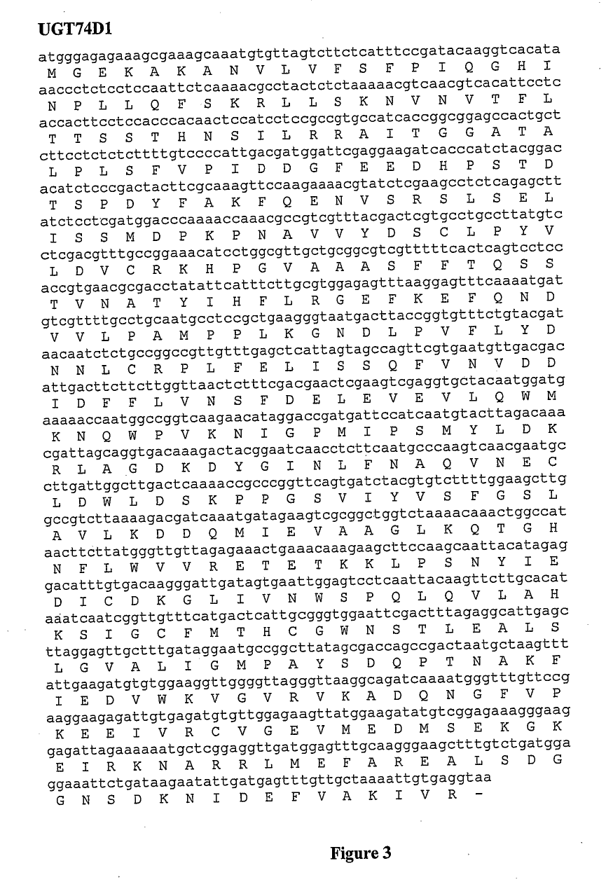

FIG. 3 Nucleic acid and amino acid sequence of UGT74D1;

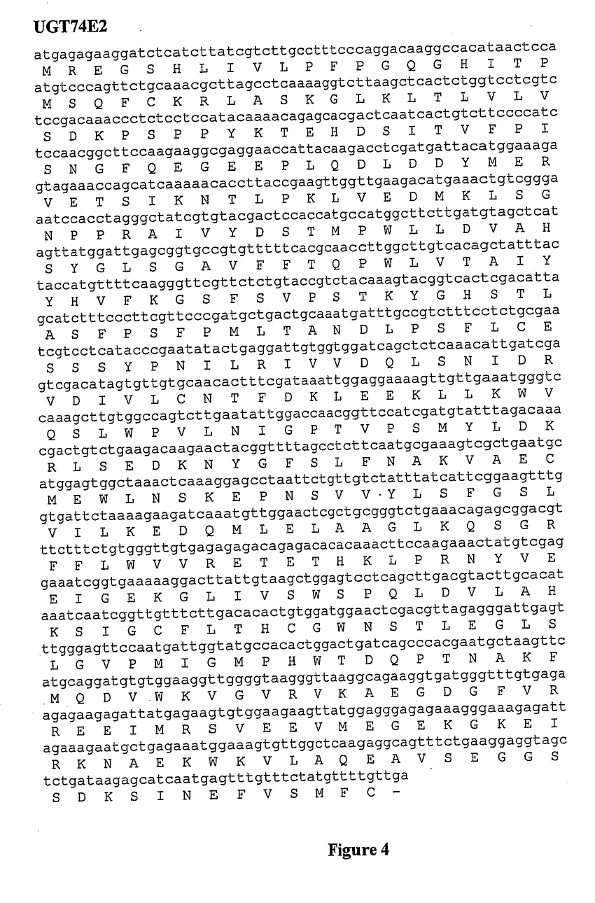

FIG. 4 Nucleic acid and amino acid sequence of UGT74E2;

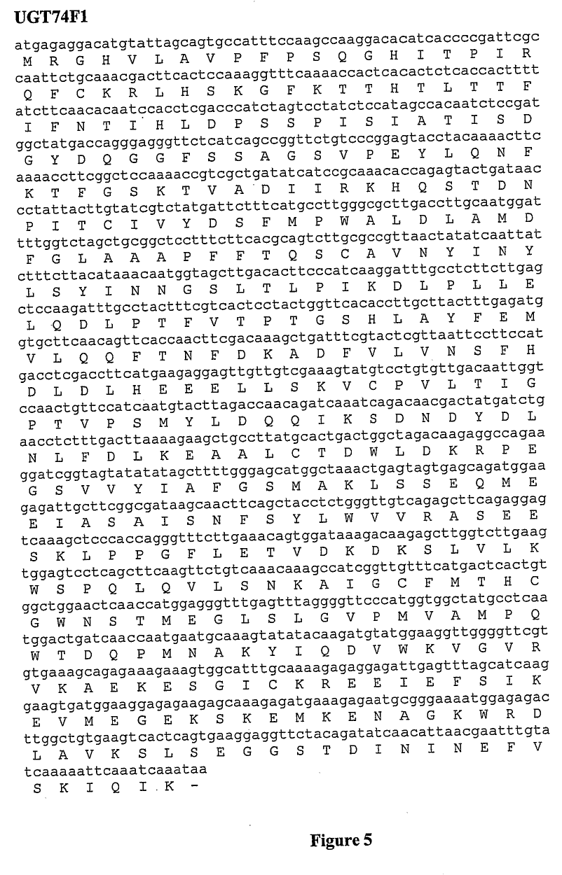

FIG. 5 Nucleic acid and amino acid sequence of UGT74F1;

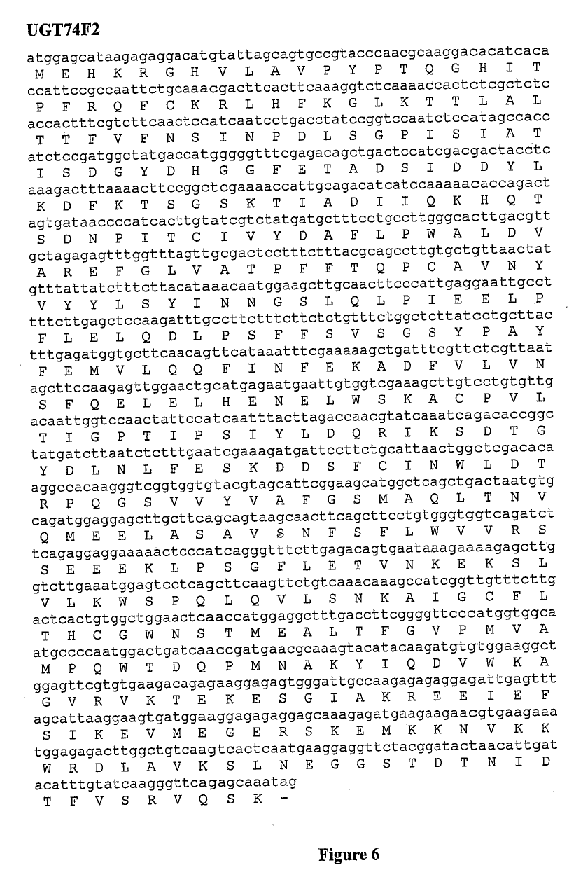

FIG. 6 Nucleic acid and amino acid sequence of UGT74E2;

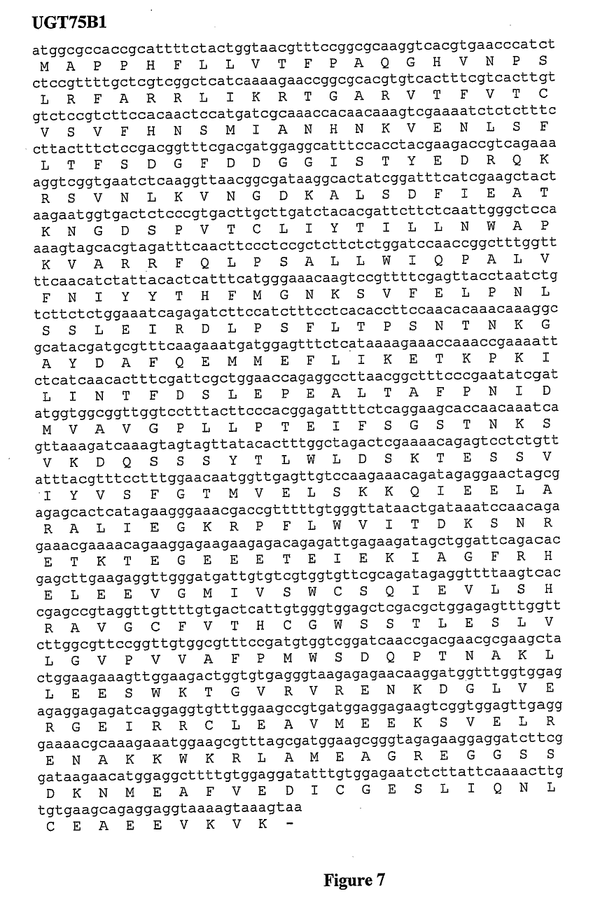

FIG. 7 Nucleic acid and amino acid sequence of UGT75B1;

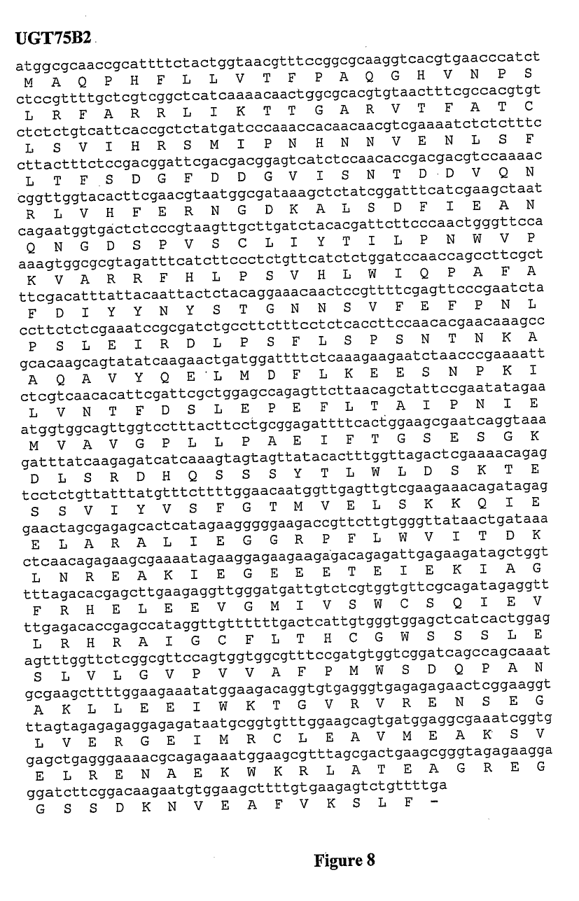

FIG. 8 Nucleic acid and amino acid sequence of UGT75B2;

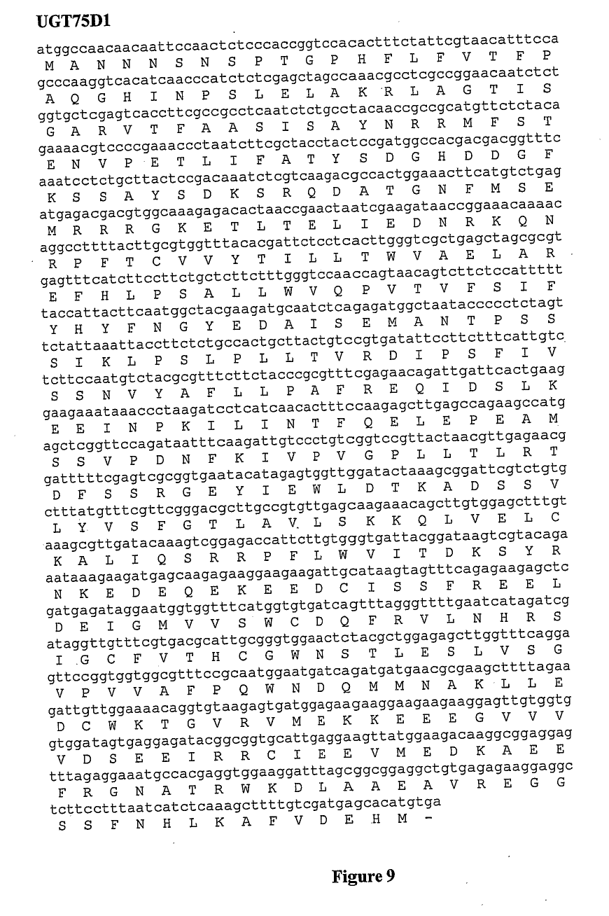

FIG. 9 Nucleic acid and amino acid sequence of UGT75D1;

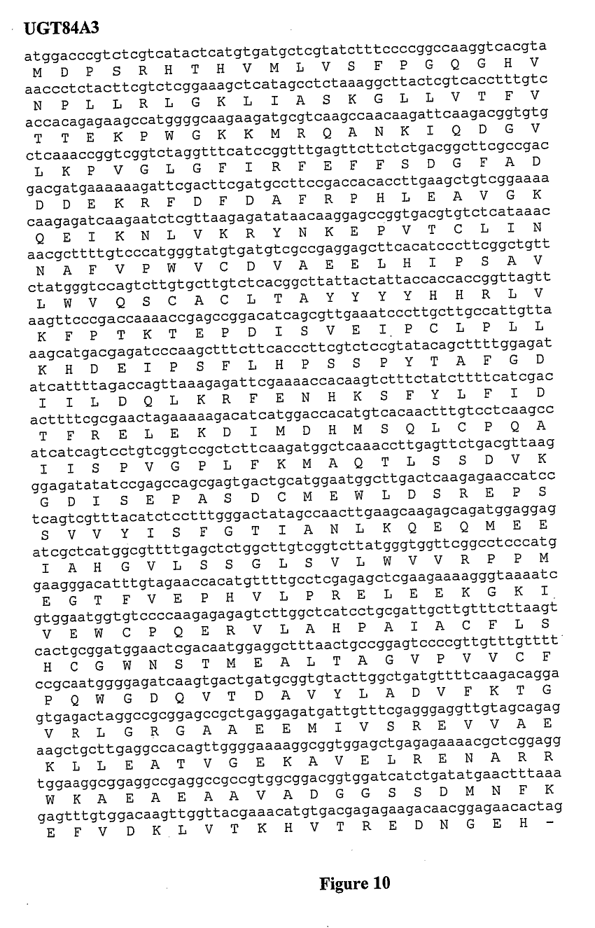

FIG. 10 Nucleic acid and amino acid sequence of UGT84A3;

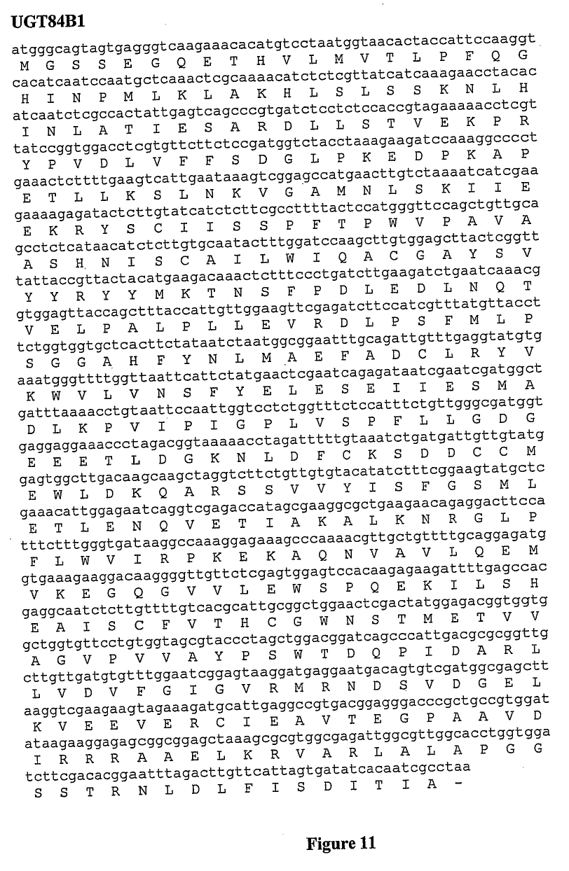

FIG. 11 Nucleic acid and amino acid sequence of UGT84B1;

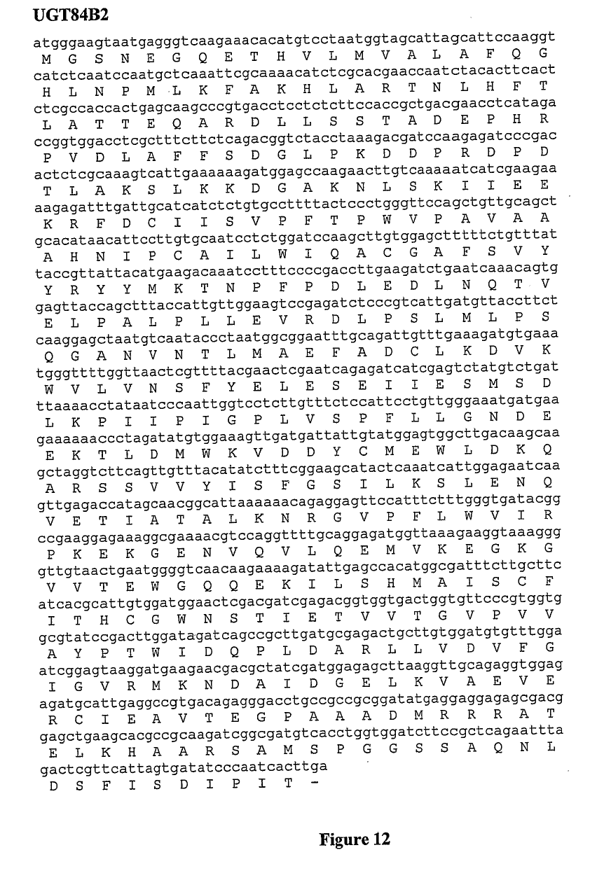

FIG. 12 Nucleic acid and amino acid sequence of UGT84B2;

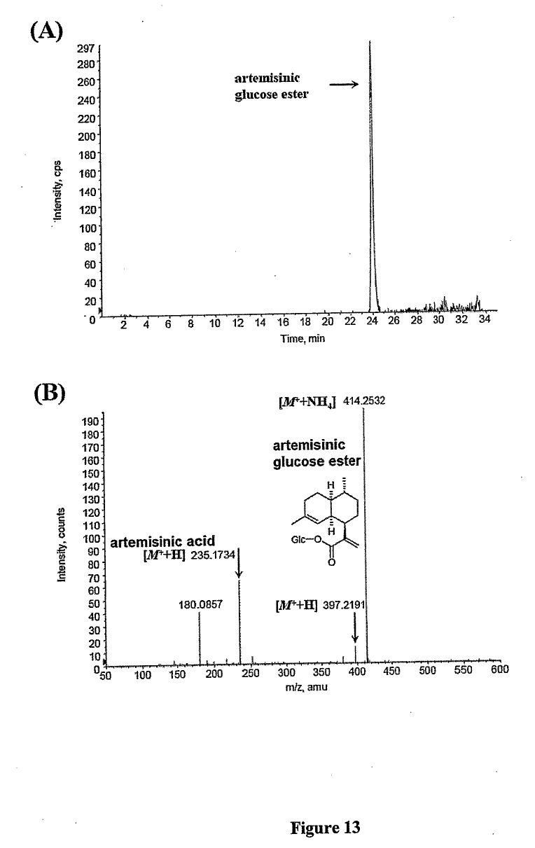

FIG. 13 LC-MS analysis of artemisinic acid glucose ester. (A) HPLC analysis. (B) MS analysis;

FIG. 14 Nucleic acid sequence of an amorphadeine synthase;

FIG. 15 Nucleic acid sequence of a cytochrome P450;

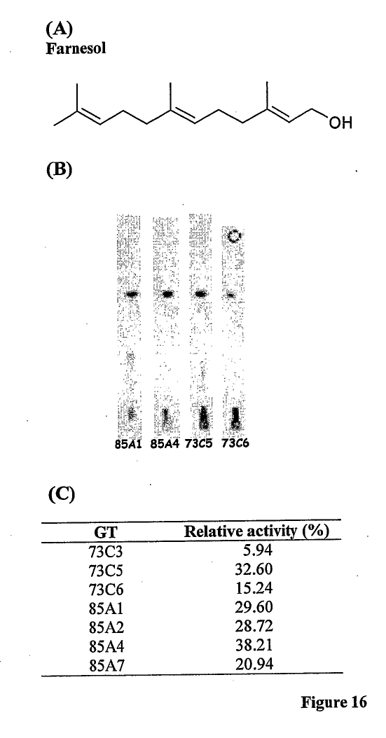

FIG. 16 (A) Chemical structure of farnesol. (B) TLC analysis of representative GTs capable of glucosylating farnesol. (C) Relative activity of the GTs towards farnesol.

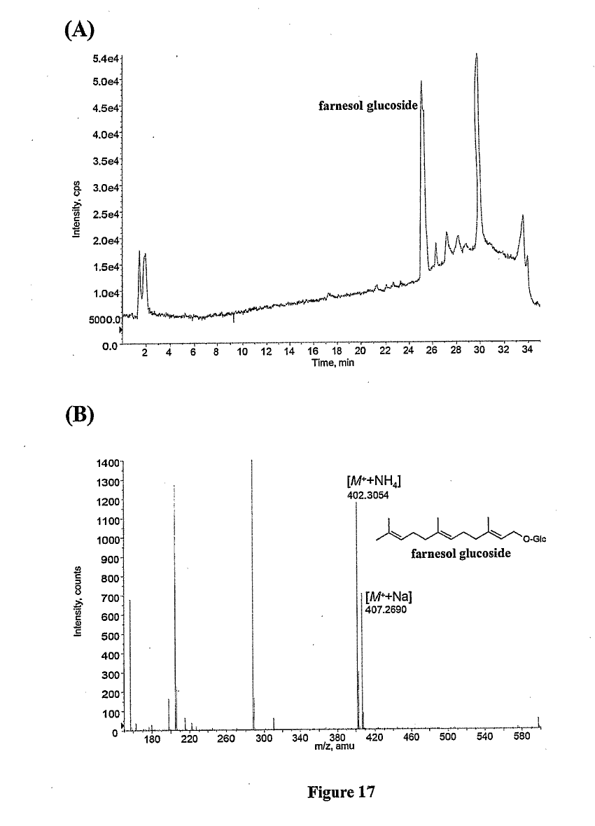

FIG. 17 LC-MS analysis of farnesol glucoside. (A) HPLC analysis. (B) MS analysis.

FIG. 18 DNA and amino acid sequence of UGT73C3;

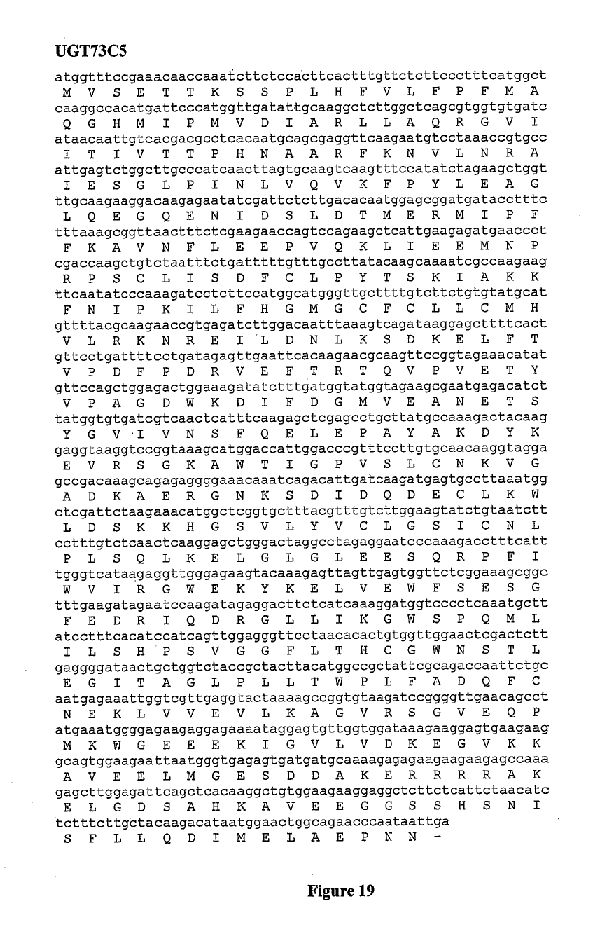

FIG. 19 DNA and amino acid sequence of UGT73C5;

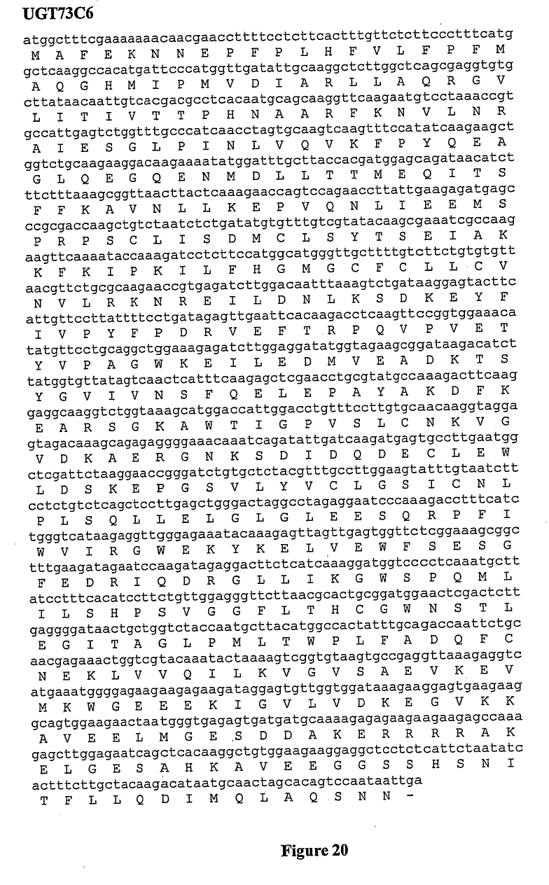

FIG. 20 DNA and amino acid sequence of UGT73C6;

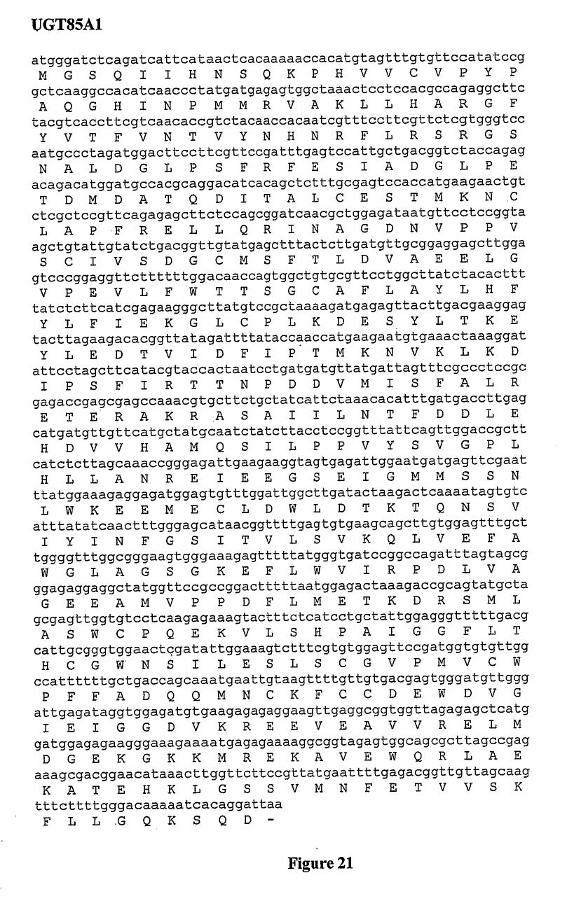

FIG. 21 DNA and amino acid sequence of UGT85A1;

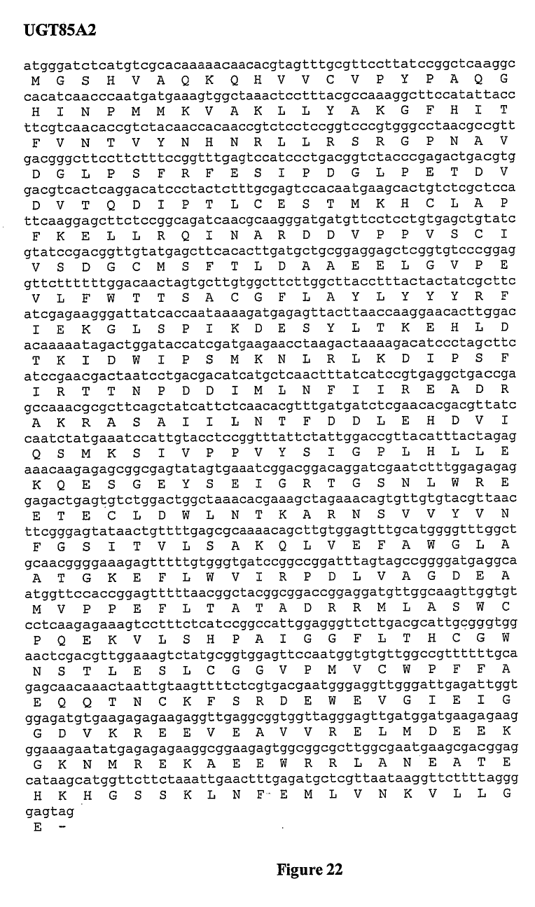

FIG. 22 DNA and amino acid sequence of UGT85A2;

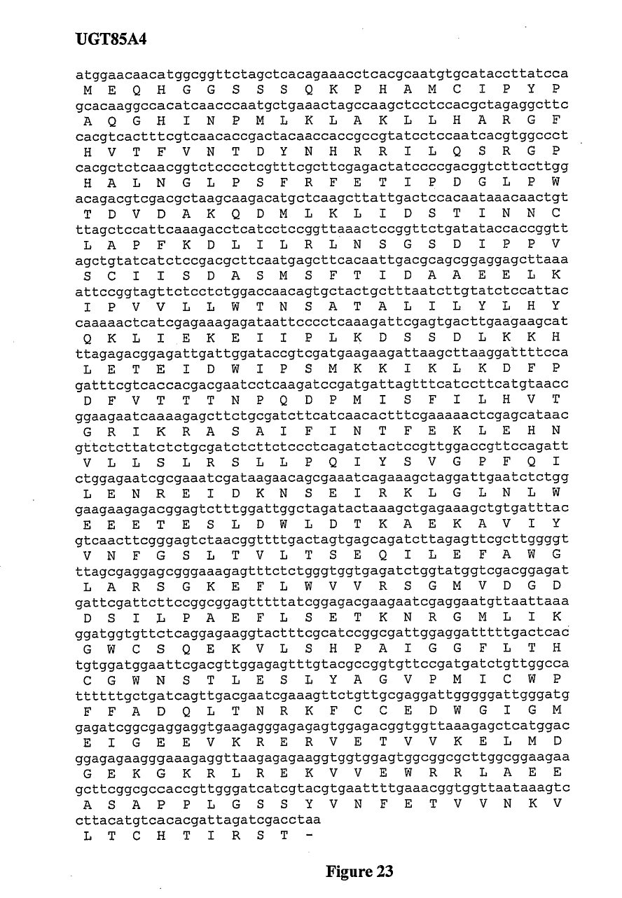

FIG. 23 DNA and amino acid sequence of UGT85A4; and

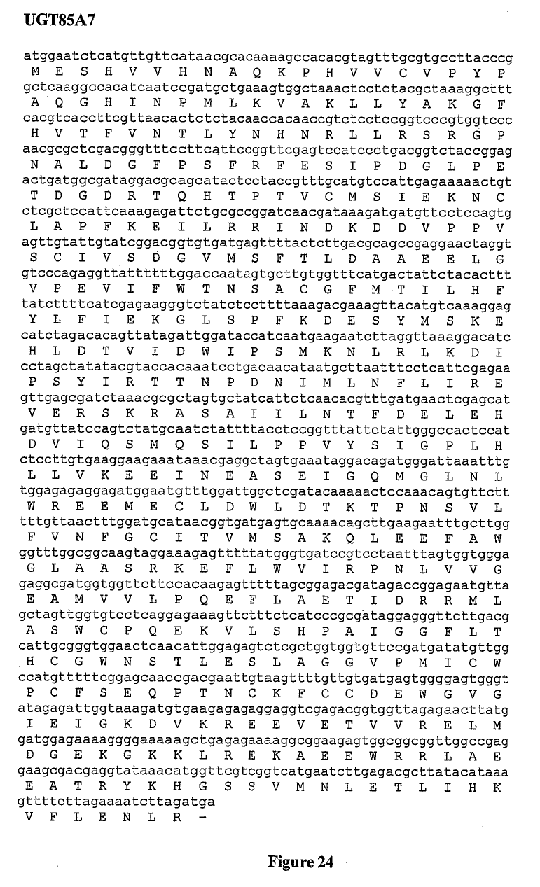

FIG. 24 DNA and amino acid sequence of UGT85A7.

### MATERIALS AND METHODS

Recombinant GTs Expression and Purification

Recombinant GTs were expressed as fusion proteins with glutathione-S-transferase (GST) attached to the N-terminus of the GTs. The GST gene fusion vector pGEX-2T (Amersham Biotech) containing the cDNA of GTs was transformed into E. coil BL21 for recombinant protein expression. The bacterial cells were grown in 75 ml of 2×YT medium containing 50 μg/ml ampicillin at 20° C. until A₆₀₀ reading reaches 1.0. The culture was then incubated with 1 mM isopropyl-1-thio-β-D-galactopyranoside for 24 h at 20° C. Cells were harvested (5000×g for 5 min), resuspended (5 ml of ice-cold phosphate-buffered saline), disrupted by lysozyme (1 mg/ml) and centrifuged again (40000×g for 15 min). The supernatant was mixed with 100 μl of 50% glutathione-coupled Sepharose at room temperature for 30 min. The beads were washed with phosphate buffer saline, and the absorbed proteins were eluted with 20 mM reduced-form glutathione according to the manufacturer's instructions. The protein concentration was determined using the Bradford method and bovine serum albumin as reference.

TLC Analysis of the Enzyme Activity

Each reaction mix (20 μl) contained 100 mM TRIS-HCl (pH 7.0), 3.7 μM ¹⁴C UDP-glucose (11.6 GBq/mmol, Amersham), 1 mM artemisinic acid and 300 ng of enzyme. The reaction was carried out at 30° C. for 2 h. The reaction mix was stored at −20° C. before TLC analysis.

The reaction mixtures were loaded on to Silica gel 60 TLC plates. The TLC analysis was carried out in a solvent system consisting of ethylacetate/acetone/dichloromethane/methanol/water (20:15:6:5:4, v/v/v/v/v). The plates were dried and exposed to phosphor-imaging screens (Molecular Dynamics) for 24 h. The screens were read using a Molecular Imager FX scanner (BioRad) supplied with Quantity One software (BioRad). The amount of ¹⁴C UDP-glucose transferred by the enzymes to the substrates was calculated using a regression equation obtained by analysing ¹⁴C UDP-glucose standards ranged between 0.008-0.555 kBq with the TLC method described above.

HPLC Analysis of Artemisinic Acid and the Glucoside

Reaction mix for HPLC analysis was performed in 200 μl volume containing 100 mM TRIS-HCl (pH 7.0), 2.5 mM UDP-glucose, 1 mM artemisinic acid and 1 μg of enzyme. The reaction was incubated at 30° C. for 2 h, and stored at −20° C. prior to HPLC analysis.

Reverse phase HPLC (SpectraSYSTEM HPLC systems and UV6000LP photodiode array detector, TermoQuest) was carried out using a Columbus 5-μm C18 column (250×4.6 mm, Phenomenex) at a flow rate of 1 ml/min with a linear gradient of 10-50% solvent A (methanol) against solvent B (10 mM ammonium acetate) over 10 min, followed by a linear gradient 10-50% A over 20 min against B. The column was then washed with 100% A for 5 min. The chromatography was monitored at 210 nm.

HPLC-MS Analysis of Glucoside

The glucoside formed in the enzymatic reaction was confirmed using an Agilent 1100 Series HPLC system (Agilente Technologies) coupled with a QStar hybrid quadrupole-TOF mass spectrometer (Applied Biosystems). The HPLC was performed with a Columbus 5μ C₁₈ column (150×3.2 mm, Phenomenex) at a flow rate of 0.5 ml/min following the conditions described in the previous section. The MS analysis was carried out in a positive ion mode. Total ion current and ion traces for specific [M+H⁺], [M+NH₄⁺] and [M+Na⁺] adducts ions were used to detect the compounds. MS-MS analysis was performed on the specific ions using different collision energies.

1. A microbial cell wherein the microbial cell is transformed with a vector that includes a nucleic acid molecule selected from the group consisting of:
i) a nucleic acid molecule comprising a nucleic acid sequence as represented in SEQ ID NO: 1-11 and 23-29;
ii) a nucleic acid molecule that hybridizes under stringent hybridization conditions to a nucleic acid molecule as represented in SEQ ID NO: 1-11 and 23-29 and that encodes a glycosyltransferase that glycosylates a sesquiterpenoid;
iii) a nucleic acid molecule that encodes a polypeptide comprising an amino acid sequence as represented in SEQ ID NO: 12-22 and 30-36; and
iv) a nucleic acid molecule that consists of a nucleic acid sequence as represented in SEQ ID NO: 1-11 and 23-29.

2. (canceled)

3. A cell according to claim 1 wherein said microbial cell is transformed with a nucleic acid molecule that encodes a polypeptide wherein said polypeptide is an amorphadeine synthase that catalyses the conversion of farnesyl diphosphate to amorpha-4,11-diene.

4. A cell according to claim 3 wherein said nucleic acid molecule comprises a nucleic acid sequence as represented in SEQ ID NO: 37, or a nucleic acid molecule that hybridises to the nucleic acid molecule in SEQ ID NO: 37 and encodes a polypeptide that is an amorphadeine synthase.

5. A cell according to claim 1 wherein said microbial cell is transformed with a nucleic acid molecule that encodes a polypeptide wherein said polypeptide is a cytochrome P450 that catalyses the conversion of amorpha-4,11-diene to artemisinic acid.

6. A cell according to claim 5 wherein said nucleic acid molecule comprises a nucleic acid sequence as represented in SEQ ID NO: 38, or a nucleic acid molecule that hybridises to the nucleic acid molecule in SEQ ID NO: 38 and encodes a polypeptide that is a P450.

7-9. (canceled)

10. A cell according to claim 1 wherein said vector is an expression vector and said nucleic acid molecule encoding said glycosyltransferase is operably linked to a promoter.

11-12. (canceled)

13. A process for the glycosylation of a sesquiterpenoid comprising the steps of:
i) forming a preparation that includes a cell according to claim 1 and a sesquiterpenoid;
ii) cultivating said preparation under conditions that allow the glycosylation of said sesquiterpenoid with a sugar; and
iii) isolating and purifying said glycosylated sesquiterpenoid from said cell and/or the surrounding cell growth medium.

14. A process according to claim 13 wherein said sesquiterpenoid is artemisinic acid or farnesol.

15-16. (canceled)

17. A transgenic plant wherein said plant is genetically modified by transfection with a vector that includes a nucleic acid molecule selected from the group consisting of:
i) a nucleic acid molecule comprising a nucleic acid sequence as represented in SEQ ID NO: 1-11 and 23-29;
ii) a nucleic acid molecule that hybridizes under stringent hybridization conditions to a nucleic acid molecule as represented in SEQ ID NO: 1-11 and 23-29 and that encodes a glycosyltransferase that glycosylates artemisinic acid; and
iii) a nucleic acid molecule that encodes a polypeptide comprising an amino acid sequence as represented in SEQ ID NO: 12-22 and 30-36.

18. (canceled)

19. A plant according to claim 17 wherein said plant is of the genus Artemesia spp.

20. (canceled)

21. An isolated artemisinic acid ester comprising a sugar pendant group to provide glycosylated artemisinic acid.

22. An ester according to claim 21 wherein artemisinic acid has the structure

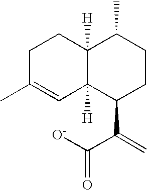

23-26. (canceled)

27. A composition comprising a glycosylated artemisinic acid according to claim 21.

28. A composition according to claim 27 wherein said composition is a pharmaceutical composition.

29. (canceled)

30. A method to treat a disorder selected from the group consisting of: a fungal infection, a bacterial infection and a cancer, comprising administering an effective amount of glycosylated artemisinic acid according to claim 21 to an animal.

31. (canceled)

32. A method according to claim 30 wherein said treatment is the topical application of glycosylated artemisinic acid.

33. (canceled)

34. An isolated farnesol ester comprising a sugar pendant group to provide glycosylated farnesol.

35. An ester according to claim 34 wherein farnesol has the structure

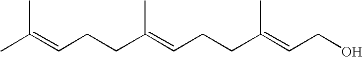

36-38. (canceled)

39. A composition comprising a glycosylated farnesol according to claim 34.

40-42. (canceled)

43. A process for the glycosylation of a sesquiterpenoid comprising the steps of:
i) providing a transgenic plant or a seed transfected with a nucleic acid molecule comprising a nucleic acid sequence selected from the group consisting of:
a) a nucleic acid sequence as represented in SEQ ID NO: 1-11 and 23-29;
b) a nucleic acid molecule that hybridizes under stringent hybridization conditions to a nucleic acid molecule as represented in SEQ ID NO: 1-11 and 23-29 and that encodes a glycosyltransferase that glycosylates a sesquiterpenoid; and
c) a nucleic acid molecule that encodes a polypeptide comprising an amino acid sequence as represented in SEQ ID NO: 12-22 and 30-36;
ii) cultivating said plant or seed under conditions that allow the glycosylation of a sesquiterpenoid with a sugar; and
iii) isolating and purifying said glycosylated sesquiterpenoid from said plant and/or said seed.


SEQUENCE LISTING (38 sequences)

SEQ ID NO: 1 - DNA, 1464 bp, Arabidopsis thaliana
atggcggaaacaactcccaaagtgaaaggccacgtcgtaatcttaccatacccagttcaaggccacctaaacccaatggttcaattcgctaaacgtctagtctccaaaaacgtcaaagtcacaatcgccaccactacctacaccgcctcctcaatcacaacaccatcactctccgtcgaaccaatctccgatggattcgatttcatccccataggtatccccggtttcagcgtcgatacttactcagaatccttcaagctcaacggatccgaaaccctaactctcctaatcgagaaattcaaatccacagattcaccaatcgattgcttaatctacgattcgtttcttccttggggacttgaagttgctagatctatggaactttcagctgcttctttcttcactaataatctcactgtttgttctgtgttgcgtaaattctctaacggtgactttcctcttcccgctgatcctaattcggcgccgtttcgtatccgtggcttaccgtctttgagctacgatgagttaccttcgtttgtgggacgtcattggttgactcatcctgagcatggcagagttcttctgaatcagtttcctaaccatgaaaatgctgattggttattcgttaatggctttgaagggttagaagaaacacaagtaagagttttgattctactataaagtttgaaactttatgttacattgttgaattgaaattagaactgttgttttgattaggattgtgaaaatggtgagtctgatgcaatgaaggcgacgttgatcggaccgatgattccatcggcttatcttgatgatcggatggaagatgataaagactatggtgcgagtctgttgaaaccgatatcgaaggagtgtatggagtggcttgagactaagcaggctcagtcagtagcatttgtttcgtttggttcgtttgggattctctttgagaagcaacttgcagaggtagctattgcgctacaagaatcggatttgaacttcttgtgggtgattaaagaagctcatatagcgaaattgcctgaagggtttgtggaatcgactaaagatagagccttgttggtttcttggtgtaaccagcttgaggttttagctcatgaatcgataggttgctttttgactcattgtggttggaactctacgttggaagggttgagtttgggagttccgatggttggtgtgcctcagtggagtgatcagatgaatgatgctaagtttgtggaggaagtttggaaagttgggtatagagcgaaagaggaagctggggaagtaatcgtgaagagtgaagaattggtgaggtgtttgaaaggagtgatggaaggagagagtagtgtgaagattagagagagttcgaagaagtggaaagatttggctgtgaaggcaatgagtgaaggaggaagctctgatcgaagcattaacgagtttatagagagtttagggaagtaa

SEQ ID NO: 2 - DNA, 1371 bp, Arabidopsis thaliana
atgggagagaaagcgaaagcaaatgtgttagtcttctcatttccgatacaaggtcacataaaccctctcctccaattctcaaaacgcctactctctaaaaacgtcaacgtcacattcctcaccacttcctccacccacaactccatcctccgccgtgccatcaccggcggagccactgctcttcctctctcttttgtccccattgacgatggattcgaggaagatcacccatctacggacacatctcccgactacttcgcaaagttccaagaaaacgtatctcgaagcctctcagagcttatctcctcgatggacccaaaaccaaacgccgtcgtttacgactcgtgcctgccttatgtcctcgacgtttgccggaaacatcctggcgttgctgcggcgtcgtttttcactcagtcctccaccgtgaacgcgacctatattcatttcttgcgtggagagtttaaggagtttcaaaatgatgtcgttttgcctgcaatgcctccgctgaagggtaatgacttaccggtgtttctgtacgataacaatctctgccggccgttgtttgagctcattagtagccagttcgtgaatgttgacgacattgacttcttcttggttaactctttcgacgaactcgaagtcgaggtgctacaatggatgaaaaaccaatggccggtcaagaacataggaccgatgattccatcaatgtacttagacaaacgattagcaggtgacaaagactacggaatcaacctcttcaatgcccaagtcaacgaatgccttgattggcttgactcaaaaccgcccggttcagtgatctacgtgtcttttggaagcttggccgtcttaaaagacgatcaaatgatagaagtcgcggctggtctaaaacaaactggccataacttcttatgggttgttagagaaactgaaacaaagaagcttccaagcaattacatagaggacatttgtgacaagggattgatagtgaattggagtcctcaattacaagttcttgcacataaatcaatcggttgtttcatgactcattgcgggtggaattcgactttagaggcattgagcttaggagttgctttgataggaatgccggcttatagcgaccagccgactaatgctaagtttattgaagatgtgtggaaggttggggttagggttaaggcagatcaaaatgggtttgttccgaaggaagagattgtgagatgtgttggagaagttatggaagatatgtcggagaaagggaaggagattagaaaaaatgctcggaggttgatggagtttgcaagggaagctttgtctgatggaggaaattctgataagaatattgatgagtttgttgctaaaattgtgaggtaa

SEQ ID NO: 3 - DNA, 1444 bp, Arabidopsis thaliana
atgagagaaggatctcatcttatcgtcttgcctttcccaggacaaggccacataactccaatgtcccagttctgcaaacgcttagcctcaaaaggtcttaagctcactctggtcctcgtctccgacaaaccctctcctccatacaaaacagagcacgactcaatcactgtcttccccatctccaacggcttccaagaaggcgaggaaccattacaagacctcgatgattacatggaaagagtagaaaccagcatcaaaaacaccttaccgaagttggttgaagacatgaaactgtcgggaaatccacctagggctatcgtgtacgactccaccatgccatggcttcttgatgtagctcatagttatggattgagcggtgccgtgtttttcacgcaaccttggcttgtcacagctatttactaccatgttttcaagggttcgttctctgtaccgtctacaaagtacggtcactcgacattagcatctttcccttcgttcccgatgctgactgcaaatgatttgccgtctttcctctgcgaatcgtcctcatacccgaatatactgaggattgtggtggatcagctctcaaacattgatcgagtcgacatagtgttgtgcaacactttcgataaattggaggaaaaggtacagaatataaatccatatagaggaacatgtctctgtcttttgtaggaagtgttttaagttttattttctctgcttgtagttgttgaaatgggtccaaagcttgtggccagtcttgaatattggaccaacggttccatcgatgtatttagacaaacgactgtctgaagacaagaactacggttttagcctcttcaatgcgaaagtcgctgaatgcatggagtggctaaactcaaaggagcctaattctgttgtctatttatcattcggaagtttggtgattctaaaagaagatcaaatgttggaactcgctgcgggtctgaaacagagcggacgtttctttctgtgggttgtgagagagacagagacacacaaacttccaagaaactatgtcgaggaaatcggtgaaaaaggacttattgtaagctggagtcctcagcttgacgtacttgcacataaatcaatcggttgtttcttgacacactgtggatggaactcgacgttagagggattgagtttgggagttccaatgattggtatgccacactggactgatcagcccacgaatgctaagttcatgcaggatgtgtggaaggttggggtaagggttaaggcagaaggtgatgggtttgtgagaagagaagagattatgagaagtgtggaagaagttatggagggagagaaagggaaagagattagaaagaatgctgagaaatggaaagtgttggctcaagaggcagtttctgaaggaggtagctctgataagagcatcaatgagtttgtttctatgttttgttga

SEQ ID NO: 4 - DNA, 1341 bp, Arabidopsis thaliana
atgagaggacatgtattagcagtgccatttccaagccaaggacacatcaccccgattcgccaattctgcaaacgacttcactccaaaggtttcaaaaccactcacactctcaccacttttatcttcaacacaatccacctcgacccatctagtcctatctccatagccacaatctccgatggctatgaccagggagggttctcatcagccggttctgtcccggagtacctacaaaacttcaaaaccttcggctccaaaaccgtcgctgatatcatccgcaaacaccagagtactgataaccctattacttgtatcgtctatgattctttcatgccttgggcgcttgaccttgcaatggattttggtctagctgcggctcctttcttcacgcagtcttgcgccgttaactatatcaattatctttcttacataaacaatggtagcttgacacttcccatcaaggatttgcctcttcttgagctccaagatttgcctactttcgtcactcctactggttcacaccttgcttactttgagatggtgcttcaacagttcaccaacttcgacaaagctgatttcgtactcgttaattccttccatgacctcgaccttcatgaagaggagttgttgtcgaaagtatgtcctgtgttgacaattggtccaactgttccatcaatgtacttagaccaacagatcaaatcagacaacgactatgatctgaacctctttgacttaaaagaagctgccttatgcactgactggctagacaagaggccagaaggatcggtagtatatatagcttttgggagcatggctaaactgagtagtgagcagatggaagagattgcttcggcgataagcaacttcagctacctctgggttgtcagagcttcagaggagtcaaagctcccaccagggtttcttgaaacagtggataaagacaagagcttggtcttgaagtggagtcctcagcttcaagttctgtcaaacaaagccatcggttgtttcatgactcactgtggctggaactcaaccatggagggtttgagtttaggggttcccatggtggctatgcctcaatggactgatcaaccaatgaatgcaaagtatatacaagatgtatggaaggttggggttcgtgtgaaagcagagaaagaaagtggcatttgcaaaagagaggagattgagtttagcatcaaggaagtgatggaaggagagaagagcaaagagatgaaagagaatgcgggaaaatggagagacttggctgtgaagtcactcagtgaaggaggttctacagatatcaacattaacgaatttgtatcaaaaattcaaatcaaataa

SEQ ID NO: 5 - DNA, 1350 bp, Arabidopsis thaliana
atggagcataagagaggacatgtattagcagtgccgtacccaacgcaaggacacatcacaccattccgccaattctgcaaacgacttcacttcaaaggtctcaaaaccactctcgctctcaccactttcgtcttcaactccatcaatcctgacctatccggtccaatctccatagccaccatctccgatggctatgaccatgggggtttcgagacagctgactccatcgacgactacctcaaagactttaaaacttccggctcgaaaaccattgcagacatcatccaaaaacaccagactagtgataaccccatcacttgtatcgtctatgatgctttcctgccttgggcacttgacgttgctagagagtttggtttagttgcgactcctttctttacgcagccttgtgctgttaactatgtttattatctttcttacataaacaatggaagcttgcaacttcccattgaggaattgccttttcttgagctccaagatttgccttctttcttctctgtttctggctcttatcctgcttactttgagatggtgcttcaacagttcataaatttcgaaaaagctgatttcgttctcgttaatagcttccaagagttggaactgcatgagaatgaattgtggtcgaaagcttgtcctgtgttgacaattggtccaactattccatcaatttacttagaccaacgtatcaaatcagacaccggctatgatcttaatctctttgaatcgaaagatgattccttctgcattaactggctcgacacaaggccacaagggtcggtggtgtacgtagcattcggaagcatggctcagctgactaatgtgcagatggaggagcttgcttcagcagtaagcaacttcagcttcctgtgggtggtcagatcttcagaggaggaaaaactcccatcagggtttcttgagacagtgaataaagaaaagagcttggtcttgaaatggagtcctcagcttcaagttctgtcaaacaaagccatcggttgtttcttgactcactgtggctggaactcaaccatggaggctttgaccttcggggttcccatggtggcaatgccccaatggactgatcaaccgatgaacgcaaagtacatacaagatgtgtggaaggctggagttcgtgtgaagacagagaaggagagtgggattgccaagagagaggagattgagtttagcattaaggaagtgatggaaggagagaggagcaaagagatgaagaagaacgtgaagaaatggagagacttggctgtcaagtcactcaatgaaggaggttctacggatactaacattgatacatttgtatcaagggttcagagcaaatag

SEQ ID NO: 6 - DNA, 1410 bp, Arabidopsis thaliana
atggcgccaccgcattttctactggtaacgtttccggcgcaaggtcacgtgaacccatctctccgttttgctcgtcggctcatcaaaagaaccggcgcacgtgtcactttcgtcacttgtgtctccgtcttccacaactccatgatcgcaaaccacaacaaagtcgaaaatctctctttccttactttctccgacggtttcgacgatggaggcatttccacctacgaagaccgtcagaaaaggtcggtgaatctcaaggttaacggcgataaggcactatcggatttcatcgaagctactaagaatggtgactctcccgtgacttgcttgatctacacgattcttctcaattgggctccaaaagtagcacgtagatttcaacttccctccgctcttctctggatccaaccggctttggttttcaacatctattacactcatttcatgggaaacaagtccgttttcgagttacctaatctgtcttctctggaaatcagagatcttccatctttcctcacaccttccaacacaaacaaaggcgcatacgatgcgtttcaagaaatgatggagtttctcataaaagaaaccaaaccgaaaattctcatcaacactttcgattcgctggaaccagaggccttaacggctttcccgaatatcgatatggtggcggttggtcctttacttcccacggagattttctcaggaagcaccaacaaatcagttaaagatcaaagtagtagttatacactttggctagactcgaaaacagagtcctctgttatttacgtttcctttggaacaatggttgagttgtccaagaaacagatagaggaactagcgagagcactcatagaagggaaacgaccgtttttgtgggttataactgataaatccaacagagaaacgaaaacagaaggagaagaagagacagagattgagaagatagctggattcagacacgagcttgaagaggttgggatgattgtgtcgtggtgttcgcagatagaggttttaagtcaccgagccgtaggttgttttgtgactcattgtgggtggagctcgacgctggagagtttggttcttggcgttccggttgtggcgtttccgatgtggtcggatcaaccgacgaacgcgaagctactggaagaaagttggaagactggtgtgagggtaagagagaacaaggatggtttggtggagagaggagagatcaggaggtgtttggaagccgtgatggaggagaagtcggtggagttgagggaaaacgcaaagaaatggaagcgtttagcgatggaagcgggtagagaaggaggatcttcggataagaacatggaggcttttgtggaggatatttgtggagaatctcttattcaaaacttgtgtgaagcagaggaggtaaaagtaaagtaa

SEQ ID NO: 7 - DNA, 1368 bp, Arabidopsis thaliana
atggcgcaaccgcattttctactggtaacgtttccggcgcaaggtcacgtgaacccatctctccgttttgctcgtcggctcatcaaaacaactggcgcacgtgtaactttcgccacgtgtctctctgtcattcaccgctctatgatcccaaaccacaacaacgtcgaaaatctctctttccttactttctccgacggattcgacgacggagtcatctccaacaccgacgacgtccaaaaccggttggtacacttcgaacgtaatggcgataaagctctatcggatttcatcgaagctaatcagaatggtgactctcccgtaagttgcttgatctacacgattcttcccaactgggttccaaaagtggcgcgtagatttcatcttccctctgttcatctctggatccaaccagccttcgctttcgacatttattacaattactctacaggaaacaactccgttttcgagttcccgaatctaccttctctcgaaatccgcgatctgccttctttcctctcaccttccaacacgaacaaagccgcacaagcagtatatcaagaactgatggattttctcaaagaagaatctaacccgaaaattctcgtcaacacattcgattcgctggagccagagttcttaacagctattccgaatatagaaatggtggcagttggtcctttacttcctgcggagattttcactggaagcgaatcaggtaaagatttatcaagagatcatcaaagtagtagttatacactttggttagactcgaaaacagagtcctctgttatttatgtttcttttggaacaatggttgagttgtcgaagaaacagatagaggaactagcgagagcactcatagaagggggaagaccgttcttgtgggttataactgataaactcaacagagaagcgaaaatagaaggagaagaagagacagagattgagaagatagctggttttagacacgagcttgaagaggttgggatgattgtctcgtggtgttcgcagatagaggttttgagacaccgagccataggttgttttttgactcattgtgggtggagctcatcactggagagtttggttctcggcgttccagtggtggcgtttccgatgtggtcggatcagccagcaaatgcgaagcttttggaagaaatatggaagacaggtgtgagggtgagagagaactcggaaggtttagtagagagaggagagataatgcggtgtttggaagcagtgatggaggcgaaatcggtggagctgagggaaaacgcagagaaatggaagcgtttagcgactgaagcgggtagagaaggaggatcttcggacaagaatgtggaagcttttgtgaagagtctgttttga

SEQ ID NO: 8 - DNA, 1425 bp, Arabidopsis thaliana
atggccaacaacaattccaactctcccaccggtccacactttctattcgtaacatttccagcccaaggtcacatcaacccatctctcgagctagccaaacgcctcgccggaacaatctctggtgctcgagtcaccttcgccgcctcaatctctgcctacaaccgccgcatgttctctacagaaaacgtccccgaaaccctaatcttcgctacctactccgatggccacgacgacggtttcaaatcctctgcttactccgacaaatctcgtcaagacgccactggaaacttcatgtctgagatgagacgacgtggcaaagagacactaaccgaactaatcgaagataaccggaaacaaaacaggccttttacttgcgtggtttacacgattctcctcacttgggtcgctgagctagcgcgtgagtttcatcttccttctgctcttctttgggtccaaccagtaacagtcttctccattttttaccattacttcaatggctacgaagatgcaatctcagagatggctaataccccctctagttctattaaattaccttctctgccactgcttactgtccgtgatattccttctttcattgtctcttccaatgtctacgcgtttcttctacccgcgtttcgagaacagattgattcactgaaggaagaaataaaccctaagatcctcatcaacactttccaagagcttgagccagaagccatgagctcggttccagataatttcaagattgtccctgtcggtccgttactaacgttgagaacggatttttcgagtcgcggtgaatacatagagtggttggatactaaagcggattcgtctgtgctttatgtttcgttcgggacgcttgccgtgttgagcaagaaacagcttgtggagctttgtaaagcgttgatacaaagtcggagaccattcttgtgggtgattacggataagtcgtacagaaataaagaagatgagcaagagaaggaagaagattgcataagtagtttcagagaagagctcgatgagataggaatggtggtttcatggtgtgatcagtttagggttttgaatcatagatcgataggttgtttcgtgacgcattgcgggtggaactctacgctggagagcttggtttcaggagttccggtggtggcgtttccgcaatggaatgatcagatgatgaacgcgaagcttttagaagattgttggaaaacaggtgtaagagtgatggagaagaaggaagaagaaggagttgtggtggtggatagtgaggagatacggcggtgcattgaggaagttatggaagacaaggcggaggagtttagaggaaatgccacgaggtggaaggatttagcggcggaggctgtgagagaaggaggctcttcctttaatcatctcaaagcttttgtcgatgagcacatgtga

SEQ ID NO: 9 - DNA, 1440 bp, Arabidopsis thaliana
atggacccgtctcgtcatactcatgtgatgctcgtatctttccccggccaaggtcacgtaaaccctctacttcgtctcggaaagctcatagcctctaaaggcttactcgtcacctttgtcaccacagagaagccatggggcaagaagatgcgtcaagccaacaagattcaagacggtgtgctcaaaccggtcggtctaggtttcatccggtttgagttcttctctgacggcttcgccgacgacgatgaaaaaagattcgacttcgatgccttccgaccacaccttgaagctgtcggaaaacaagagatcaagaatctcgttaagagatataacaaggagccggtgacgtgtctcataaacaacgcttttgtcccatgggtatgtgatgtcgccgaggagcttcacatcccttcggctgttctatgggtccagtcttgtgcttgtctcacggcttattactattaccaccaccggttagttaagttcccgaccaaaaccgagccggacatcagcgttgaaatcccttgcttgccattgttaaagcatgacgagatcccaagctttcttcacccttcgtctccgtatacagcttttggagatatcattttagaccagttaaagagattcgaaaaccacaagtctttctatcttttcatcgacacttttcgcgaactagaaaaagacatcatggaccacatgtcacaactttgtcctcaagccatcatcagtcctgtcggtccgctcttcaagatggctcaaaccttgagttctgacgttaagggagatatatccgagccagcgagtgactgcatggaatggcttgactcaagagaaccatcctcagtcgtttacatctcctttgggactatagccaacttgaagcaagagcagatggaggagatcgctcatggcgttttgagctctggcttgtcggtcttatgggtggttcggcctcccatggaagggacatttgtagaaccacatgttttgcctcgagagctcgaagaaaagggtaaaatcgtggaatggtgtccccaagagagagtcttggctcatcctgcgattgcttgtttcttaagtcactgcggatggaactcgacaatggaggctttaactgccggagtccccgttgtttgttttccgcaatggggagatcaagtgactgatgcggtgtacttggctgatgttttcaagacaggagtgagactaggccgcggagccgctgaggagatgattgtttcgagggaggttgtagcagagaagctgcttgaggccacagttggggaaaaggcggtggagctgagagaaaacgctcggaggtggaaggcggaggccgaggccgccgtggcggacggtggatcatctgatatgaactttaaagagtttgtggacaagttggttacgaaacatgtgacgagagaagacaacggagaacactag

SEQ ID NO: 10 - DNA, 1371 bp, Arabidopsis thaliana
atgggcagtagtgagggtcaagaaacacatgtcctaatggtaacactaccattccaaggtcacatcaatccaatgctcaaactcgcaaaacatctctcgttatcatcaaagaacctacacatcaatctcgccactattgagtcagcccgtgatctcctctccaccgtagaaaaacctcgttatccggtggacctcgtgttcttctccgatggtctacctaaagaagatccaaaggcccctgaaactcttttgaagtcattgaataaagtcggagccatgaacttgtctaaaatcatcgaagaaaagagatactcttgtatcatctcttcgccttttactccatgggttccagctgttgcagcctctcataacatctcttgtgcaatactttggatccaagcttgtggagcttactcggtttattaccgttactacatgaagacaaactctttccctgatcttgaagatctgaatcaaacggtggagttaccagctttaccattgttggaagttcgagatcttccatcgtttatgttaccttctggtggtgctcacttctataatctaatggcggaatttgcagattgtttgaggtatgtgaaatgggttttggttaattcattctatgaactcgaatcagagataatcgaatcgatggctgatttaaaacctgtaattccaattggtcctctggtttctccatttctgttgggcgatggtgaggaggaaaccctagacggtaaaaacctagatttttgtaaatctgatgattgttgtatggagtggcttgacaagcaagctaggtcttctgttgtgtacatatctttcggaagtatgctcgaaacattggagaatcaggtcgagaccatagcgaaggcgctgaagaacagaggacttccatttctttgggtgataaggccaaaggagaaagcccaaaacgttgctgttttgcaggagatggtgaaagaaggacaaggggttgttctcgagtggagtccacaagagaagattttgagccacgaggcaatctcttgttttgtcacgcattgcggctggaactcgactatggagacggtggtggctggtgttcctgtggtagcgtaccctagctggacggatcagcccattgacgcgcggttgcttgttgatgtgtttggaatcggagtaaggatgaggaatgacagtgtcgatggcgagcttaaggtcgaagaagtagaaagatgcattgaggccgtgacggagggacccgctgccgtggatataagaaggagagcggcggagctaaagcgcgtggcgagattggcgttggcacctggtggatcttcgacacggaatttagacttgttcattagtgatatcacaatcgcctaa

SEQ ID NO: 11 - DNA, 1353 bp, Arabidopsis thaliana
atgggaagtaatgagggtcaagaaacacatgtcctaatggtagcattagcattccaaggtcatctcaatccaatgctcaaattcgcaaaacatctcgcacgaaccaatctacacttcactctcgccaccactgagcaagcccgtgacctcctctcttccaccgctgacgaacctcatagaccggtggacctcgctttcttctcagacggtctacctaaagacgatccaagagatcccgacactctcgcaaagtcattgaaaaaagatggagccaagaacttgtcaaaaatcatcgaagaaaagagatttgattgcatcatctctgtgccttttactccctgggttccagctgttgcagctgcacataacattccttgtgcaatcctctggatccaagcttgtggagctttttctgtttattaccgttattacatgaagacaaatcctttccccgaccttgaagatctgaatcaaacagtggagttaccagctttaccattgttggaagtccgagatctcccgtcattgatgttaccttctcaaggagctaatgtcaataccctaatggcggaatttgcagattgtttgaaagatgtgaaatgggttttggttaactcgttttacgaactcgaatcagagatcatcgagtctatgtctgatttaaaacctataatcccaattggtcctcttgtttctccattcctgttgggaaatgatgaagaaaaaaccctagatatgtggaaagttgatgattattgtatggagtggcttgacaagcaagctaggtcttcagttgtttacatatctttcggaagcatactcaaatcattggagaatcaagttgagaccatagcaacggcattaaaaaacagaggagttccatttctttgggtgatacggccgaaggagaaaggcgaaaacgtccaggttttgcaggagatggttaaagaaggtaaaggggttgtaactgaatggggtcaacaagaaaagatattgagccacatggcgatttcttgcttcatcacgcattgtggatggaactcgacgatcgagacggtggtgactggtgttcccgtggtggcgtatccgacttggatagatcagccgcttgatgcgagactgcttgtggatgtgtttggaatcggagtaaggatgaagaacgacgctatcgatggagagcttaaggttgcagaggtggagagatgcattgaggccgtgacagagggacctgccgccgcggatatgaggaggagagcgacggagctgaagcacgccgcaagatcggcgatgtcacctggtggatcttccgctcagaatttagactcgttcattagtgatatcccaatcacttga

SEQ ID NO: 12 - protein, 460 aa, Arabidopsis thaliana
MAETTPKVKGHVVILPYPVQGHLNPMVQFAKRLVSKNVKVTIATTTYTASSITTPSLSVEPISDGFDFIPIGIPGFSVDTYSESFKLNGSETLTLLIEKFKSTDSPIDCLIYDSFLPWGLEVARSMELSAASFFTNNLTVCSVLRKFSNGDFPLPADPNSAPFRIRGLPSLSYDELPSFVGRHWLTHPEHGRVLLNQFPNHENADWLFVNGFEGLEETQDCENGESDAMKATLIGPMIPSAYLDDRMEDDKDYGASLLKPISKECMEWLETKQAQSVAFVSFGSFGILFEKQLAEVAIALQESDLNFLWVIKEAHIAKLPEGFVESTKDRALLVSWCNQLEVLAHESIGCFLTHCGWNSTLEGLSLGVPMVGVPQWSDQMNDAKFVEEVWKVGYRAKEEAGEVIVKSEELVRCLKGVMEGESSVKIRESSKKWKDLAVKAMSEGGSSDRSINEFIESLGK

SEQ ID NO: 13 - protein, 456 aa, Arabidopsis thaliana
MGEKAKANVLVFSFPIQGHINPLLQFSKRLLSKNVNVTFLTTSSTHNSILRRAITGGATALPLSFVPIDDGFEEDHPSTDTSPDYFAKFQENVSRSLSELISSMDPKPNAVVYDSCLPYVLDVCRKHPGVAAASFFTQSSTVNATYIHFLRGEFKEFQNDVVLPAMPPLKGNDLPVFLYDNNLCRPLFELISSQFVNVDDIDFFLVNSFDELEVEVLQWMKNQWPVKNIGPMIPSMYLDKRLAGDKDYGINLFNAQVNECLDWLDSKPPGSVIYVSFGSLAVLKDDQMIEVAAGLKQTGHNFLWVVRETETKKLPSNYIEDICDKGLIVNWSPQLQVLAHKSIGCFMTHCGWNSTLEALSLGVALIGMPAYSDQPTNAKFIEDVWKVGVRVKADQNGFVPKEEIVRCVGEVMEDMSEKGKEIRKNARRLMEFAREALSDGGNSDKNIDEFVAKIVR

SEQ ID NO: 14 - protein, 453 aa, Arabidopsis thaliana
MREGSHLIVLPFPGQGHITPMSQFCKRLASKGLKLTLVLVSDKPSPPYKTEHDSITVFPISNGFQEGEEPLQDLDDYMERVETSIKNTLPKLVEDMKLSGNPPRAIVYDSTMPWLLDVAHSYGLSGAVFFTQPWLVTAIYYHVFKGSFSVPSTKYGHSTLASFPSFPMLTANDLPSFLCESSSYPNILRIVVDQLSNIDRVDIVLCNTFDKLEEKLLKWVQSLWPVLNIGPTVPSMYLDKRLSEDKNYGFSLFNAKVAECMEWLNSKEPNSVVYLSFGSLVILKEDQMLELAAGLKQSGRFFLWVVRETETHKLPRNYVEEIGEKGLIVSWSPQLDVLAHKSIGCFLTHCGWNSTLEGLSLGVPMIGMPHWTDQPTNAKFMQDVWKVGVRVKAEGDGFVRREEIMRSVEEVMEGEKGKEIRKNAEKWKVLAQEAVSEGGSSDKSINEFVSMFC

SEQ ID NO: 15 - protein, 449 aa, Arabidopsis thaliana
MEKMRGHVLAVPFPSQGHITPIRQFCKRLHSKGFKTTHTLTTFIFNTIHLDPSSPISIATISDGYDQGGFSSAGSVPEYLQNFKTFGSKTVADIIRKHQSTDNPITCIVYDSFMPWALDLAMDFGLAAAPFFTQSCAVNYINYLSYINNGSLTLPIKDLPLLELQDLPTFVTPTGSHLAYFEMVLQQFTNFDKADFVLVNSFHDLDLHEEELLSKVCPVLTIGPTVPSMYLDQQIKSDNDYDLNLFDLKEAALCTDWLDKRPEGSVVYIAFGSMAKLSSEQMEEIASAISNFSYLWVVRASEESKLPPGFLETVDKDKSLVLKWSPQLQVLSNKAIGCFMTHCGWNSTMEGLSLGVPMVAMPQWTDQPMNAKYIQDVWKVGVRVKAEKESGICKREEIEFSIKEVMEGEKSKEMKENAGKWRDLAVKSLSEGGSTDININEFVSKIQIK

SEQ ID NO: 16 - protein, 449 aa, Arabidopsis thaliana
MEHKRGHVLAVPYPTQGHITPFRQFCKRLHFKGLKTTLALTTFVFNSINPDLSGPISIATISDGYDHGGFETADSIDDYLKDFKTSGSKTIADIIQKHQTSDNPITCIVYDAFLPWALDVAREFGLVATPFFTQPCAVNYVYYLSYINNGSLQLPIEELPFLELQDLPSFFSVSGSYPAYFEMVLQQFINFEKADFVLVNSFQELELHENELWSKACPVLTIGPTIPSIYLDQRIKSDTGYDLNLFESKDDSFCINWLDTRPQGSVVYVAFGSMAQLTNVQMEELASAVSNFSFLWVVRSSEEEKLPSGFLETVNKEKSLVLKWSPQLQVLSNKAIGCFLTHCGWNSTMEALTFGVPMVAMPQWTDQPMNAKYIQDVWKAGVRVKTEKESGIAKREEIEFSIKEVMEGERSKEMKKNVKKWRDLAVKSLNEGGSTDTNIDTFVSRVQSK

SEQ ID NO: 17 - protein, 469 aa, Arabidopsis thaliana
MAPPHFLLVTFPAQGHVNPSLRFARRLIKRTGARVTFVTCVSVFHNSMIANHNKVENLSFLTFSDGFDDGGISTYEDRQKRSVNLKVNGDKALSDFIEATKNGDSPVTCLIYTILLNWAPKVARRFQLPSALLWIQPALVFNIYYTHFMGNKSVFELPNLSSLEIRDLPSFLTPSNTNKGAYDAFQEMMEFLIKETKPKILINTFDSLEPEALTAFPNIDMVAVGPLLPTEIFSGSTNKSVKDQSSSYTLWLDSKTESSVIYVSFGTMVELSKKQIEELARALIEGKRPFLWVITDKSNRETKTEGEEETEIEKIAGFRHELEEVGMIVSWCSQIEVLSHRAVGCFVTHCGWSSTLESLVLGVPVVAFPMWSDQPTNAKLLEESWKTGVRVRENKDGLVERGEIRRCLEAVMEEKSVELRENAKKWKRLAMEAGREGGSSDKNMEAFVEDICGESLIQNLCEAEEVKVK

SEQ ID NO: 18 - protein, 455 aa, Arabidopsis thaliana
MAQPHFLLVTFPAQGHVNPSLRFARRLIKTTGARVTFATCLSVIHRSMIPNHNNVENLSFLTFSDGFDDGVISNTDDVQNRLVHFERNGDKALSDFIEANQNGDSPVSCLIYTILPNWVPKVARRFHLPSVHLWIQPAFAFDIYYNYSTGNNSVFEFPNLPSLEIRDLPSFLSPSNTNKAAQAVYQELMDFLKEESNPKILVNTFDSLEPEFLTAIPNIEMVAVGPLLPAEIFTGSESGKDLSRDHQSSSYTLWLDSKTESSVIYVSFGTMVELSKKQIEELARALIEGGRPFLWVITDKLNREAKIEGEEETEIEKIAGFRHELEEVGMIVSWCSQIEVLRHRAIGCFLTHCGWSSSLESLVLGVPVVAFPMWSDQPANAKLLEEIWKTGVRVRENSEGLVERGEIMRCLEAVMEAKSVELRENAEKWKRLATEAGREGGSSDKNVEAFVKSLF

SEQ ID NO: 19 - protein, 474 aa, Arabidopsis thaliana
MANNNSNSPTGPHFLFVTFPAQGHINPSLELAKRLAGTISGARVTFAASISAYNRRMFSTENVPETLIFATYSDGHDDGFKSSAYSDKSRQDATGNFMSEMRRRGKETLTELIEDNRKQNRPFTCVVYTILLTWVAELAREFHLPSALLWVQPVTVFSIFYHYFNGYEDAISEMANTPSSSIKLPSLPLLTVRDIPSFIVSSNVYAFLLPAFREQIDSLKEEINPKILINTFQELEPEAMSSVPDNFKIVPVGPLLTLRTDFSSRGEYIEWLDTKADSSVLYVSFGTLAVLSKKQLVELCKALIQSRRPFLWVITDKSYRNKEDEQEKEEDCISSSEKSFDEIGMVVSWCDQFRVLNHRSIGCFVTHCGWNSTLESLVSGVPVVAFPQWNDQMTNAKLLEDCWKTGVRVMEKKEEEGVVVVDSEEIRRCIEEVMEDKAEEFRGNATRWKDLAAEAVREGGSSFNHLKAFVDEHM

SEQ ID NO: 20 - protein, 479 aa, Arabidopsis thaliana
MDPSRHTHVMLVSFPGQGHVNPLLRLGKLIASKGLLVTFVTTEKPWGKKMRQANKIQDGVLKPVGLGFIRFEFFSDGFADDDEKRFDFDAFRPHLEAVGKQEIKNLVKRYNKEPVTCLINNAFVPWVCDVAEELHIPSAVLWVQSCACLTAYYYYHHRLVKFPTKTEPDISVEIPCLPLLKHDEIPSFLHPSSPYTAFGDIILDQLKRFENHKSFYLFIDTFRELEKDIMDHMSQLCPQAIISPVGPLFKMAQTLSSDVKGDISEPASDCMEWLDSREPSSVVYISFGTIANLKQEQMEEIAHGVLSSGLSVLWVVRPPMEGTFVEPHVLPRELEEKGKIVEWCPQERVLAHPAIACFLSHCGWNSTMEALTAGVPVVCFPQWGDQVTDAVYLADVFKTGVRLGRGAAEEMIVSREVVAEKLLEATVGEKAVELRENARRWKAEAEAAVADGGSSDMNFKEFVDKLVTKHVTREDNGEH

SEQ ID NO: 21 - protein, 456 aa, Arabidopsis thaliana
MGSSEGQETHVLMVTLPFQGHINPMLKLAKHLSLSSKNLHINLATIESARDLLSTVEKPRYPVDLVFFSDGLPKEDPKAPETLLKSLNKVGAMNLSKIIEEKRYSCIISSPFTPWVPAVAASHNISCAILWIQACGAYSVYYRYYMKTNSFPDLEDLNQTVELPALPLLEVRDLPSFMLPSGGAHFYNLMAEFADCLRYVKWVLVNSFYELESEIIESMADLKPVIPIGPLVSPFLLGDGEEETLDGKNLDFCKSDDCCMEWLDKQARSSVVYISFGSMLETLENQVETIAKALKNRGLPFLWVIRPKEKAQNVAVLQEMVKEGQGVVLEWSPQEKILSHEAISCFVTHCGWNSTMETVVAGVPVVAYPSWTDQPIDARLLVDVFGIGVRMRNDSVDGELKVEEVERCIEAVTEGPAAVDIRRRAAELKRVARLALAPGGSSTRNLDLFISDITIA

SEQ ID NO: 22 - protein, 450 aa, Arabidopsis thaliana
MGSNEGQETHVLMVALAFQGHLNPMLKFAKHLARTNLHFTLATTEQARDLLSSTADEPHRPVDLAFFSDGLPKDDPRDPDTLAKSLKKDGAKNLSKIIEEKRFDCIISVPFTPWVPAVAAAHNIPCAILWIQACGAFSVYYRYYMKTNPFPDLEDLNQTVELPALPLLEVRDLPSLMLPSQGANVNTLMAEFADCLKDVKWVLVNSFYELESEIIESMSDLKPIIPIGPLVSPFLLGNDEEKTLDMWKVDDYCMEWLDKQARSSVVYISFGSILKSLENQVETIATALKNRGVPFLWVIRPKEKGENVQVLQEMVKEGKGVVTEWGQQEKILSHMAISCFITHCGWNSTIETVVTGVPVVAYPTWIDQPLDARLLVDVFGIGVRMKNDAIDGELKVAEVERCIEAVTEGPAAADMRRRATELKHAARSAMSPGGSSAQNLDSFISDIPIT

SEQ ID NO: 23 - DNA, 1491 bp, Arabidopsis thaliana
atggctacggaaaaaacccaccaatttcatccttctcttcactttgtcctcttccctttcatggctcaaggccacatgattcccatgattgatattgcaagactcttggctcagcgtggtgtgaccataacaattgtcacgacacctcacaacgcagcaaggtttaagaatgtcctaaaccgagcgatcgagtctggcttggccatcaacatactgcatgtgaagtttccatatcaagagtttggtttgccagaaggaaaagagaatatagattcgttagactcaacggagttgatggtacctttcttcaaagcggtgaacttgcttgaagatccggtcatgaagctcatggaagagatgaaacctagacctagctgtctaatttctgattggtgtttgccttatacaagcataatcgccaagaacttcaatataccaaagatagttttccacggcatgggttgctttaatcttttgtgtatgcatgttctacgcagaaacttagagatcctagagaatgtaaagtcggatgaagagtatttcttggttcctagttttcctgatagagttgaatttacaaagcttcaacttcctgtgaaagcaaatgcaagtggagattggaaagagataatggatgaaatggtaaaagcagaatacacatcctatggtgtgatcgtcaacacatttcaggagttggagccaccttatgtcaaagactacaaagaggcaatggatggaaaagtatggtccattggacccgtttccttgtgtaacaaggcaggtgcagacaaagctgagaggggaagcaaggccgccattgatcaagatgagtgtcttcaatggcttgattctaaagaagaaggttcggtgctctatgtttgccttggaagtatatgtaatcttcctttgtctcagctcaaggagctggggctaggccttgaggaatctcgaagatcttttatttgggtcataagaggttcggaaaagtataaagaactatttgagtggatgttggagagcggttttgaagaaagaatcaaagagagaggacttctcattaaagggtgggcacctcaagtccttatcctttcacatccttccgttggaggattcctgacacactgtggatggaactcgactctcgaaggaatcacctcaggcattccactgatcacttggccgctgtttggagaccaattctgcaaccaaaaactggtcgttcaagtactaaaagccggtgtaagtgccggggttgaagaagtcatgaaatggggagaagaagataaaataggagtgttagtggataaagaaggagtgaaaaaggctgtggaagaattgatgggtgatagtgatgatgcaaaagagaggagaagaagagtcaaagagcttggagaattagctcacaaagctgtggaaaaaggaggctcttctcattctaacatcacactcttgctacaagacataatgcaactagcacaattcaagaattga

SEQ ID NO: 24 - DNA, 1488 bp, Arabidopsis thaliana
atggtttccgaaacaaccaaatcttctccacttcactttgttctcttccctttcatggctcaaggccacatgattcccatggttgatattgcaaggctcttggctcagcgtggtgtgatcataacaattgtcacgacgcctcacaatgcagcgaggttcaagaatgtcctaaaccgtgccattgagtctggcttgcccatcaacttagtgcaagtcaagtttccatatctagaagctggtttgcaagaaggacaagagaatatcgattctcttgacacaatggagcggatgatacctttctttaaagcggttaactttctcgaagaaccagtccagaagctcattgaagagatgaaccctcgaccaagctgtctaatttctgatttttgtttgccttatacaagcaaaatcgccaagaagttcaatatcccaaagatcctcttccatggcatgggttgcttttgtcttctgtgtatgcatgttttacgcaagaaccgtgagatcttggacaatttaaagtcagataaggagcttttcactgttcctgattttcctgatagagttgaattcacaagaacgcaagttccggtagaaacatatgttccagctggagactggaaagatatctttgatggtatggtagaagcgaatgagacatcttatggtgtgatcgtcaactcatttcaagagctcgagcctgcttatgccaaagactacaaggaggtaaggtccggtaaagcatggaccattggacccgtttccttgtgcaacaaggtaggagccgacaaagcagagaggggaaacaaatcagacattgatcaagatgagtgccttaaatggctcgattctaagaaacatggctcggtgctttacgtttgtcttggaagtatctgtaatcttcctttgtctcaactcaaggagctgggactaggcctagaggaatcccaaagacctttcatttgggtcataagaggttgggagaagtacaaagagttagttgagtggttctcggaaagcggctttgaagatagaatccaagatagaggacttctcatcaaaggatggtcccctcaaatgcttatcctttcacatccatcagttggagggttcctaacacactgtggttggaactcgactcttgaggggataactgctggtctaccgctacttacatggccgctattcgcagaccaattctgcaatgagaaattggtcgttgaggtactaaaagccggtgtaagatccggggttgaacagcctatgaaatggggagaagaggagaaaataggagtgttggtggataaagaaggagtgaagaaggcagtggaagaattaatgggtgagagtgatgatgcaaaagagagaagaagaagagccaaagagcttggagattcagctcacaaggctgtggaagaaggaggctcttctcattctaacatctctttcttgctacaagacataatggaactggcagaacccaataattga

SEQ ID NO: 25 - DNA, 1488 bp, Arabidopsis thaliana
atggctttcgaaaaaaacaacgaaccttttcctcttcactttgttctcttccctttcatggctcaaggccacatgattcccatggttgatattgcaaggctcttggctcagcgaggtgtgcttataacaattgtcacgacgcctcacaatgcagcaaggttcaagaatgtcctaaaccgtgccattgagtctggtttgcccatcaacctagtgcaagtcaagtttccatatcaagaagctggtctgcaagaaggacaagaaaatatggatttgcttaccacgatggagcagataacatctttctttaaagcggttaacttactcaaagaaccagtccagaaccttattgaagagatgagcccgcgaccaagctgtctaatctctgatatgtgtttgtcgtatacaagcgaaatcgccaagaagttcaaaataccaaagatcctcttccatggcatgggttgcttttgtcttctgtgtgttaacgttctgcgcaagaaccgtgagatcttggacaatttaaagtctgataaggagtacttcattgttccttattttcctgatagagttgaattcacaagacctcaagttccggtggaaacatatgttcctgcaggctggaaagagatcttggaggatatggtagaagcggataagacatcttatggtgttatagtcaactcatttcaagagctcgaacctgcgtatgccaaagacttcaaggaggcaaggtctggtaaagcatggaccattggacctgtttccttgtgcaacaaggtaggagtagacaaagcagagaggggaaacaaatcagatattgatcaagatgagtgccttgaatggctcgattctaaggaaccgggatctgtgctctacgtttgccttggaagtatttgtaatcttcctctgtctcagctccttgagctgggactaggcctagaggaatcccaaagacctttcatctgggtcataagaggttgggagaaatacaaagagttagttgagtggttctcggaaagcggctttgaagatagaatccaagatagaggacttctcatcaaaggatggtcccctcaaatgcttatcctttcacatccttctgttggagggttcttaacgcactgcggatggaactcgactcttgaggggataactgctggtctaccaatgcttacatggccactatttgcagaccaattctgcaacgagaaactggtcgtacaaatactaaaagtcggtgtaagtgccgaggttaaagaggtcatgaaatggggagaagaagagaagataggagtgttggtggataaagaaggagtgaagaaggcagtggaagaactaatgggtgagagtgatgatgcaaaagagagaagaagaagagccaaagagcttggagaatcagctcacaaggctgtggaagaaggaggctcctctcattctaatatcactttcttgctacaagacataatgcaactagcacagtccaataattga

SEQ ID NO: 26 - DNA, 1470 bp, Arabidopsis thaliana
atgggatctcagatcattcataactcacaaaaaccacatgtagtttgtgttccatatccggctcaaggccacatcaaccctatgatgagagtggctaaactcctccacgccagaggcttctacgtcaccttcgtcaacaccgtctacaaccacaatcgtttccttcgttctcgtgggtccaatgccctagatggacttccttcgttccgatttgagtccattgctgacggtctaccagagacagacatggatgccacgcaggacatcacagctctttgcgagtccaccatgaagaactgtctcgctccgttcagagagcttctccagcggatcaacgctggagataatgttcctccggtaagctgtattgtatctgacggttgtatgagctttactcttgatgttgcggaggagcttggagtcccggaggttcttttttggacaaccagtggctgtgcgttcctggcttatctacacttttatctcttcatcgagaagggcttatgtccgctaaaagatgagagttacttgacgaaggagtacttagaagacacggttatagattttataccaaccatgaagaatgtgaaactaaaggatattcctagcttcatacgtaccactaatcctgatgatgttatgattagtttcgccctccgcgagaccgagcgagccaaacgtgcttctgctatcattctaaacacatttgatgaccttgagcatgatgttgttcatgctatgcaatctatcttacctccggtttattcagttggaccgcttcatctcttagcaaaccgggagattgaagaaggtagtgagattggaatgatgagttcgaatttatggaaagaggagatggagtgtttggattggcttgatactaagactcaaaatagtgtcatttatatcaactttgggagcataacggttttgagtgtgaagcagcttgtggagtttgcttggggtttggcgggaagtgggaaagagtttttatgggtgatccggccagatttagtagcgggagaggaggctatggttccgccggactttttaatggagactaaagaccgcagtatgctagcgagttggtgtcctcaagagaaagtactttctcatcctgctattggagggtttttgacgcattgcgggtggaactcgatattggaaagtctttcgtgtggagttccgatggtgtgttggccattttttgctgaccagcaaatgaattgtaagttttgttgtgacgagtgggatgttgggattgagataggtggagatgtgaagagagaggaagttgaggcggtggttagagagctcatggatggagagaagggaaagaaaatgagagaaaaggcggtagagtggcagcgcttagccgagaaagcgacggaacataaacttggttcttccgttatgaattttgagacggttgttagcaagtttcttttgggacaaaaatcacaggattaa

SEQ ID NO: 27 - DNA, 1446 bp, Arabidopsis thaliana
atgggatctcatgtcgcacaaaaacaacacgtagtttgcgttccttatccggctcaaggccacatcaacccaatgatgaaagtggctaaactcctttacgccaaaggcttccatattaccttcgtcaacaccgtctacaaccacaaccgtctcctccggtcccgtgggcctaacgccgttgacgggcttccttctttccggtttgagtccatccctgacggtctacccgagactgacgtggacgtcactcaggacatccctactctttgcgagtccacaatgaagcactgtctcgctccattcaaggagcttctccggcagatcaacgcaagggatgatgttcctcctgtgagctgtatcgtatccgacggttgtatgagcttcacacttgatgctgcggaggagctcggtgtcccggaggttcttttttggacaactagtgcttgtggcttcttggcttacctttactactatcgcttcatcgagaagggattatcaccaataaaagatgagagttacttaaccaaggaacacttggacacaaaaatagactggataccatcgatgaagaacctaagactaaaagacatccctagcttcatccgaacgactaatcctgacgacatcatgctcaactttatcatccgtgaggctgaccgagccaaacgcgcttcagctatcattctcaacacgtttgatgatctcgaacacgacgttatccaatctatgaaatccattgtacctccggtttattctattggaccgttacatttactagagaaacaagagagcggcgagtatagtgaaatcggacggacaggatcgaatctttggagagaggagactgagtgtctggactggctaaacacgaaagctagaaacagtgttgtgtacgttaacttcgggagtataactgttttgagcgcaaaacagcttgtggagtttgcatggggtttggctgcaacggggaaagagtttttgtgggtgatccggccggatttagtagccggggatgaggcaatggttccaccggagtttttaacggctacggcggaccggaggatgttggcaagttggtgtcctcaagagaaagtcctttctcatccggccattggagggttcttgacgcattgcgggtggaactcgacgttggaaagtctatgcggtggagttccaatggtgtgttggccgttttttgcagagcaacaaactaattgtaagttttctcgtgacgaatgggaggttgggattgagattggtggagatgtgaagagagaagaggttgaggcggtggttagggagttgatggatgaagagaagggaaagaatatgagagagaaggcggaagagtggcggcgcttggcgaatgaagcgacggagcataagcatggttcttctaaattgaactttgagatgctcgttaataaggttcttttaggggagtag

SEQ ID NO: 28 - DNA, 1470 bp, Arabidopsis thaliana
atggaacaacatggcggttctagctcacagaaacctcacgcaatgtgcataccttatccagcacaaggccacatcaacccaatgctgaaactagccaagctcctccacgctagaggcttccacgtcactttcgtcaacaccgactacaaccaccgccgtatcctccaatcacgtggccctcacgctctcaacggtctcccctcgtttcgcttcgagactatccccgacggtcttccttggacagacgtcgacgctaagcaagacatgctcaagcttattgactccacaataaacaactgtttagctccattcaaagacctcatcctccggttaaactccggttctgatataccaccggttagctgtatcatctccgacgcttcaatgagcttcacaattgacgcagcggaggagcttaaaattccggtagttctcctctggaccaacagtgctactgctttaatcttgtatctccattaccaaaaactcatcgagaaagagataattcccctcaaagattcgagtgacttgaagaagcatttagagacggagattgattggataccgtcgatgaagaagattaagcttaaggattttccagatttcgtcaccacgacgaatcctcaagatccgatgattagtttcatccttcatgtaaccggaagaatcaaaagagcttctgcgatcttcatcaacactttcgaaaaactcgagcataacgttctcttatctctgcgatctcttctccctcagatctactccgttggaccgttccagattctggagaatcgcgaaatcgataagaacagcgaaatcagaaagctaggattgaatctctgggaagaagagacggagtctttggattggctagatactaaagctgagaaagctgtgatttacgtcaacttcgggagtctaacggttttgactagtgagcagatcttagagttcgcttggggtttagcgaggagcgggaaagagtttctctgggtggtgagatctggtatggtcgacggagatgattcgattcttccggcggagtttttatcggagacgaagaatcgaggaatgttaattaaaggatggtgttctcaggagaaggtactttcgcatccggcgattggaggatttttgactcactgtggatggaattcgacgttggagagtttgtacgccggtgttccgatgatctgttggccattttttgctgatcagttgacgaatcgaaagttctgttgcgaggattgggggattgggatggagatcggcgaggaggtgaagagggagagagtggagacggtggttaaagagctcatggacggagagaagggaaagaggttaagagagaaggtggtggagtggcggcgcttggcggaagaagcttcggcgccaccgttgggatcatcgtacgtgaattttgaaacggtggttaataaagtccttacatgtcacacgattagatcgacctaa

SEQ ID NO: 29 - DNA, 1464 bp, Arabidopsis thaliana
atggaatctcatgttgttcataacgcacaaaagccacacgtagtttgcgtgccttacccggctcaaggccacatcaatccgatgctgaaagtggctaaactcctctacgctaaaggctttcacgtcaccttcgttaacactctctacaaccacaaccgtctcctccggtcccgtggtcccaacgcgctcgacgggtttccttcattccggttcgagtccatccctgacggtctaccggagactgatggcgataggacgcagcatactcctaccgtttgcatgtccattgagaaaaactgtctcgctccattcaaagagattctgcgccggatcaacgataaagatgatgttcctccagtgagttgtattgtatcggacggtgtgatgagttttactcttgacgcagccgaggaactaggtgtcccagaggttattttttggaccaatagtgcttgtggtttcatgactattctacacttttatcttttcatcgagaagggtctatctccttttaaagacgaaagttacatgtcaaaggagcatctagacacagttatagattggataccatcaatgaagaatcttaggttaaaggacatccctagctatatacgtaccacaaatcctgacaacataatgcttaatttcctcattcgagaagttgagcgatctaaacgcgctagtgctatcattctcaacacgtttgatgaactcgagcatgatgttatccagtctatgcaatctattttacctccggtttattctattgggccactccatctccttgtgaaggaagaaataaacgaggctagtgaaataggacagatgggattaaatttgtggagagaggagatggaatgtttggattggctcgatacaaaaactccaaacagtgttctttttgttaactttggatgcataacggtgatgagtgcaaaacagcttgaagaatttgcttggggtttggcggcaagtaggaaagagtttttatgggtgatccgtcctaatttagtggtgggagaggcgatggtggttcttccacaagagtttttagcggagacgatagaccggagaatgttagctagttggtgtcctcaggagaaagttctttctcatcccgcgataggagggttcttgacgcattgcgggtggaactcaacattggagagtctcgctggtggtgttccgatgatatgttggccatgtttttcggagcaaccgacgaattgtaagttttgttgtgatgagtggggagtgggtatagagattggtaaagatgtgaagagagaggaggtcgagacggtggttagagaacttatggatggagaaaaggggaaaaagctgagagaaaaggcggaagagtggcggcggttggccgaggaagcgacgaggtataaacatggttcgtcggtcatgaatcttgagacgcttatacataaagttttcttagaaaatcttagatga

SEQ ID NO: 30 - protein, 496 aa, Arabidopsis thaliana
MATEKTHQFHPSLHFVLFPFMAQGHMIPMIDIARLLAQRGVTITIVTTPHNAARFKNVLNRAIESGLAINILHVKFPYQEFGLPEGKENIDSLDSTELMVPFFKAVNLLEDPVMKLMEEMKPRPSCLISDWCLPYTSIIAKNFNIPKIVFHGMGCFNLLCMHVLRRNLEILENVKSDEEYFLVPSFPDRVEFTKLQLPVKANASGDWKEIMDEMVKAEYTSYGVIVNTFQELEPPYVKDYKEAMDGKVWSIGPVSLCNKAGADKAERGSKAAIDQDECLQWLDSKEEGSVLYVCLGSICNLPLSQLKELGLGLEESRRSFIWVIRGSEKYKELFEWMLESGFEERIKERGLLIKGWAPQVLILSHPSVGGFLTHCGWNSTLEGITSGIPLITWPLFGDQFCNQKLVVQVLKAGVSAGVEEVMKWGEEDKIGVLVDKEGVKKAVEELMGDSDDAKERRRRVKELGELAHKAVEKGGSSHSNITLLLQDIMQLAQFKN

SEQ ID NO: 31 - protein, 495 aa, Arabidopsis thaliana
MVSETTKSSPLHFVLFPFMAQGHMIPMVDIARLLAQRGVIITIVTTPHNAARFKNVLNRAIESGLPINLVQVKFPYLEAGLQEGQENIDSLDTMERMIPFFKAVNFLEEPVQKLIEEMNPRPSCLISDFCLPYTSKIAKKFNIPKILFHGMGCFCLLCMHVLRKNREILDNLKSDKELFTVPDFPDRVEFTRTQVPVETYVPAGDWKDIFDGMVEANETSYGVIVNSFQELEPAYAKDYKEVRSGKAWTIGPVSLCNKVGADKAERGNKSDIDQDECLKWLDSKKHGSVLYVCLGSICNLPLSQLKELGLGLEESQRPFIWVIRGWEKYKELVEWFSESGFEDRIQDRGLLIKGWSPQMLILSHPSVGGFLTHCGWNSTLEGITAGLPLLTWPLFADQFCNEKLVVEVLKAGVRSGVEQPMKWGEEEKIGVLVDKEGVKKAVEELMGESDDAKERRRRAKELGDSAHKAVEEGGSSHSNISFLLQDIMELAEPNN

SEQ ID NO: 32 - protein, 495 aa, Arabidopsis thaliana
MAFEKNNEPFPLHFVLFPFMAQGHMIPMVDIARLLAQRGVLITIVTTPHNAARFKNVLNRAIESGLPINLVQVKFPYQEAGLQEGQENMDLLTTMEQITSFFKAVNLLKEPVQNLIEEMSPRPSCLISDMCLSYTSEIAKKFKIPKILFHGMGCFCLLCVNVLRKNREILDNLKSDKEYFIVPYFPDRVEFTRPQVPVETYVPAGWKEILEDMVEADKTSYGVIVNSFQELEPAYAKDFKEARSGKAWTIGPVSLCNKVGVDKAERGNKSDIDQDECLEWLDSKEPGSVLYVCLGSICNLPLSQLLELGLGLEESQRPFIWVIRGWEKYKELVEWFSESGFEDRIQDRGLLIKGWSPQMLILSHPSVGGFLTHCGWNSTLEGITAGLPMLTWPLFADQFCNEKLVVQILKVGVSAEVKEVMKWGEEEKIGVLVDKEGVKKAVEELMGESDDAKERRRRAKELGESAHKAVEEGGSSHSNITFLLQDIMQLAQSNN

SEQ ID NO: 33 - protein, 489 aa, Arabidopsis thaliana
MGSQIIHNSQKPHVVCVPYPAQGHINPMMRVAKLLHARGFYVTFVNTVYNHNRFLRSRGSNALDGLPSFRFESIADGLPETDMDATQDITALCESTMKNCLAPFRELLQRINAGDNVPPVSCIVSDGCMSFTLDVAEELGVPEVLFWTTSGCAFLAYLHFYLFIEKGLCPLKDESYLTKEYLEDTVIDFIPTMKNVKLKDIPSFIRTTNPDDVMISFALRETERAKRASAIILNTFDDLEHDVVHAMQSILPPVYSVGPLHLLANREIEEGSEIGMMSSNLWKEEMECLDWLDTKTQNSVIYINFGSITVLSVKQLVEFAWGLAGSGKEFLWVIRPDLVAGEEAMVPPDFLMETKDRSMLASWCPQEKVLSHPAIGGFLTHCGWNSILESLSCGVPMVCWPFFADQQMNCKFCCDEWDVGIEIGGDVKREEVEAVVRELMDGEKGKKMREKAVEWQRLAEKATEHKLGSSVMNFETVVSKFLLGQKSQD

SEQ ID NO: 34 - protein, 481 aa, Arabidopsis thaliana
MGSHVAQKQHVVCVPYPAQGHINPMMKVAKLLYAKGFHITFVNTVYNHNRLLRSRGPNAVDGLPSFRFESIPDGLPETDVDVTQDIPTLCESTMKHCLAPFKELLRQINARDDVPPVSCIVSDGCMSFTLDAAEELGVPEVLFWTTSACGFLAYLYYYRFIEKGLSPIKDESYLTKEHLDTKIDWIPSMKNLRLKDIPSFIRTTNPDDIMLNFIIREADRAKRASAIILNTFDDLEHDVIQSMKSIVPPVYSIGPLHLLEKQESGEYSEIGRTGSNLWREETECLDWLNTKARNSVVYVNFGSITVLSAKQLVEFAWGLAATGKEFLWVIRPDLVAGDEAMVPPEFLTATADRRMLASWCPQEKVLSHPAIGGFLTHCGWNSTLESLCGGVPMVCWPFFAEQQTNCKFSRDEWEVGIEIGGDVKREEVEAVVRELMDEEKGKNMREKAEEWRRLANEATEHKHGSSKLNFEMLVNKVLLGE

SEQ ID NO: 35 - protein, 489 aa, Arabidopsis thaliana
MEQHGGSSSQKPHAMCIPYPAQGHINPMLKLAKLLHARGFHVTFVNTDYNHRRILQSRGPHALNGLPSFRFETIPDGLPWTDVDAKQDMLKLIDSTINNCLAPFKDLILRLNSGSDIPPVSCIISDASMSFTIDAAEELKIPVVLLWTNSATALILYLHYQKLIEKEIIPLKDSSDLKKHLETEIDWIPSMKKIKLKDFPDFVTTTNPQDPMISFILHVTGRIKRASAIFINTFEKLEHNVLLSLRSLLPQIYSVGPFQILENREIDKNSEIRKLGLNLWEEETESLDWLDTKAEKAVIYVNFGSLTVLTSEQILEFAWGLARSGKEFLWVVRSGMVDGDDSILPAEFLSETKNRGMLIKGWCSQEKVLSHPAIGGFLTHCGWNSTLESLYAGVPMICWPFFADQLTNRKFCCEDWGIGMEIGEEVKRERVETVVKELMDGEKGKRLREKVVEWRRLAEEASAPPLGSSYVNFETVVNKVLTCHTIRST

SEQ ID NO: 36 - protein, 487 aa, Arabidopsis thaliana
MESHVVHNAQKPHVVCVPYPAQGHINPMLKVAKLLYAKGFHVTFVNTLYNHNRLLRSRGPNALDGFPSFRFESIPDGLPETDGDRTQHTPTVCMSIEKNCLAPFKEILRRINDKDDVPPVSCIVSDGVMSFTLDAAEELGVPEVIFWTNSACGFMTILHFYLFIEKGLSPFKDESYMSKEHLDTVIDWIPSMKNLRLKDIPSYIRTTNPDNIMLNFLIREVERSKRASAIILNTFDELEHDVIQSMQSILPPVYSIGPLHLLVKEEINEASEIGQMGLNLWREEMECLDWLDTKTPNSVLFVNFGCITVMSAKQLEEFAWGLAASRKEFLWVIRPNLVVGEAMVVLPQEFLAETIDRRMLASWCPQEKVLSHPAIGGFLTHCGWNSTLESLAGGVPMICWPCFSEQPTNCKFCCDEWGVGIEIGKDVKREEVETVVRELMDGEKGKKLREKAEEWRRLAEEATRYKHGSSVMNLETLIHKVFLENLR

SEQ ID NO: 37 - DNA, 1641 bp, Artemesia annua
atgtcccttacagaagaaaaacctattcgccccattgccaactttcctccaagcatttggggagatcagtctctcatctatgaaaagcaagtagagcaaggggtggaacagatagtgaatgatttaaaaaaagaagtgcggcaactactaaaagaagctttggatattcctatgaaacatgccaatttgttgaagctgattgatgaaatccaacgccttggaataccgtatcactttgaacgggagattgatcatgcattgcaatgtatttatgaaacatatggtgataactggaatggtgaccgctcttccttatggttccgtcttatgcgaaagcaaggatattatgttacatgtgatgttttcaataactataaagacaaaaatggagcgttcaagcaatcgttagctaatgatgttgaaggtttgcttgagttgtacgaagcaacttctatgagggtacctggggagactatattagaagatgctcttggttttacacgatctcgtcttagcattatgacaaaagatgctttttctacaaaccccgctctttttaccgaaatacaacgggcactaaagcaacccctttggaaaaggttgccaagaatagaggcggcgcagtacattcctttctatcaacaacaagattctcataacaagactttacttaaacttgctaagttagagttcaatttgcttcagtcattgcacaaggaagagctcagccatgtgtgcaaatggtggaaagctttcgatatcaagaagaacgcaccttgtttaagagatagaattgttgaatgctacttttggggactaggttcaggctatgagccacagtattcccgggctagagttttcttcacaaaagctgttgctgttataactcttatagatgacacttatgatgcgtatggtacttatgaagaacttaagatctttactgaagctgttgaaaggtggtcaattacatgcttagacacacttccagaatacatgaaaccgatatacaaattattcatggatacatacacagaaatggaagaatttcttgcaaaggagggaagaacagatctatttaactgcggcaaagaatttgtgaaagagtttgttagaaacctgatggttgaagcaaattgggcaaatgagggacacataccaaccactgaagagcatgatccagttgtaatcattactggcggtgctaacctgcttacaacaacttgttatcttgacatgagtgatatattcacaaaagagtctgtcgaatgggctgtctctgcacctcctctttttagatactcaggtatacttggtcgacgcctagatgatctcatgacccacaaggccgagcaagaaagaaaacatagttcatcgagccttgaaagttatatgaaggaatataatgtcaatgaggagtatgcccaaaccttgatttacaaggaagtagaagatgtgtggaaagatataaaccgagagtacctcacaactaaaaacattccaaggccgttattgatggctgtgatctatttgtgccagtttcttgaagttcaatatgcaggaaaggataacttcacacgtatgggagacgaatacaaacatctcataaagtctctactcgtttatcctatgagtatatga

SEQ ID NO: 38 - DNA, 1488 bp, Artemisia annua
atgaagagtatactaaaagcaatggctctctcactgaccacttccattgctcttgcaacgatccttttgttcgtttacaagttcgctactcgttccaagtccaccaaaaaaagccttcctgagccatggcgacttcccattattggtcacatgcatcacttgattggtacaacgccacatcgtggggttagggatttagccagaaagtatggatctttgatgcatttacagcttggtgaagttccaacaattgtggtgtcatctccgaaatgggctaaagaggttttgacaacgtacgacattacctttgctaacaggcccgagactttaactggtgagattgttttatatcacaatacggatgttgttcttgcaccttatggtgagtactggaggcaattacgtaaaatttgcacattggagcttttgagtgttaagaaagtaaagtcatttcagtcgcttcgtgaagaggagtgttggaatttggttcaagagattaaagcttcaggttcagggagaccggttaacctttcagagaatgttttcaagttgattgcaacgatacttagtagagccgcatttgggaaagggatcaaggaccagaaagagttaacggagattgtgaaagagatactgaggcaaactggtggttttgatgtggcagatatctttccttcaaagaaatttcttcatcatctttcgggcaagagagctcggttaactagccttcgcaaaaagatcgataatttaatcgataaccttgtagctgagcatactgttaacacctccagtaaaactaacgagacactcctcgatgttcttttaaggctcaaagacagtgctgaattcccattaacatctgataacattaaagccatcattttggatatgtttggagcaggcacagacacttcctcatccacaatcgaatgggcgattccggaactcataaagtgtccgaaagcaatggagaaagtacaagcggaattgaggaaagcattgaacggaaaagaaaagatccatgaggaagacattcaagaactaagctacttgaacatggtaatcaaagaaacattgaggttgcaccctccactacccttggttctgccaagagagtgccgccaaccagtcaatttggctggatacaacatacccaataagaccaaacttattgtcaacgtctttgcgataaatagggaccctgaatattggaaagacgctgaagctttcatccctgaacgatttgaaaatagttctgcaactgtcatgggtgcagaatacgagtatcttccgtttggagctgggagaaggatgtgtcctggagccgcacttggtttagctaacgtgcagctcccgctcgctaatatactatatcatttcaactggaaactccccaatggtgtgagctatgaccagatcgacatgaccgagagctctggagccacgatgcaaagaaaggctgagttgttactcgttccaagtttctag